(19) United States
(12) Patent Application Publication
(10) Pub. No.: US 2013/0059303 A1
(43) Pub. Date: Mar. 7, 2013

(54) CDKN2A AS A PROGNOSTIC MARKER IN BLADDER CANCER

(71) Applicant: FRANCOIS RADVANYI, Fontenay-Aux-Roses (FR)

(72) Inventor: FRANCOIS RADVANYI, Fontenay-Aux-Roses (FR)

(21) Appl. No.: 13/604,990

(22) Filed: Sep. 6, 2012

Related U.S. Application Data
(60) Provisional application No. 61/531,205, filed on Sep. 6, 2011.

(51) Int. Cl. C12Q 1/68; G01N 21/64

(52) U.S. Cl. 435/6.11

(57) ABSTRACT

The present invention provides methods for predicting clinical outcome and for providing information for determining follow-up strategy of a subject affected with a non-muscle invasive bladder cancer, as well as a method for selecting a subject affected with a non-muscle invasive bladder cancer for an anti-tumoral therapy. The present invention also provides kits for implementing these methods.

### CROSS-REFERENCE TO RELATED APPLICATION

This application claims the benefit of U.S. Provisional Patent Application 61/531,205, filed Sep. 6, 2011, the disclosure of which is hereby incorporated by reference in its entirety.

### FIELD OF THE INVENTION

The present invention relates to the field of medicine, in particular of oncology. It provides a new prognostic marker in human bladder cancer.

### BACKGROUND OF THE INVENTION

Bladder cancer is the fourth cancer in men and the ninth in women in terms of incidence in Western countries. Most of these tumors (>90%) are derived from the epithelium lining the bladder cavity, the urothelium. These tumors are pathologically characterized by the depth of invasion of the bladder wall (stage), and their degree of differentiation (grade).

At first presentation approximately 50% of bladder tumors are Ta tumors, generally of low grade (Ta tumors are papillary tumors which do not invade the basement membrane), 20% are T1 tumors (T1 tumors invade the basement membrane but not the underlying smooth muscle) and 25% are muscle-invasive tumors (T2-4). Carcinoma in situ (CIS), flat high grade lesion not invading the basement membrane, is rarely found isolated; it is encountered predominantly with other urothelial tumors. CIS tumors often progress (in about 50% of cases) to T1 and then, to muscle-invasive tumors.

Clinical and molecular evidence suggests that there are two divergent pathways of bladder tumorigenesis: the Ta pathway and the carcinoma in situ pathway. The Ta pathway is characterized by a high frequency of activating mutations of the FGFR3 gene (Billerey et al., 2001), which encodes the tyrosine kinase receptor “fibroblast growth factor receptor 3”. These mutations are present in 70-75% of Ta tumors and absent in CIS (Billerey et al., 2001; Zieger et al., 2009).

Although of general good prognosis, Ta tumors have a high propensity for recurrence and can unexpectedly progress to muscle-invasive life threatening disease, therefore requiring lifelong surveillance. Using pathologic and clinical parameters, including EORTC risk scores, are not sufficient to predict accurately clinical behavior of non-muscle invasive bladder tumors. Identifying patients at high risk for progression remains a challenging issue.

Therefore, there is a great need for the identification of prognostic markers that can accurately distinguish bladder tumors of the Ta pathway associated with poor prognosis including high probability of disease progression, metastasis or decreased patient survival, from others Ta tumors. Using such markers, the practitioner can predict the patient's prognosis and then can adapt the therapeutic protocol and the follow-up of patients.

### SUMMARY OF THE INVENTION

The inventors herein demonstrated that hemizygous or homozygous CDKN2A deletions in non-muscle-invasive FGFR3-mutated bladder tumor are associated with shorter progression free survival. Therefore, they showed that CDKN2A gene can be used as a molecular predictive marker of progression in non invasive bladder cancers with an activating mutation in FGFR3 gene.

Accordingly, in a first aspect, the present invention concerns a method for predicting clinical outcome of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative of a non-muscle invasive bladder cancer with FGFR3 activating mutation with a poor prognosis. In an embodiment, a poor prognosis is a shorter progression-free survival and/or an increased metastasis occurrence and/or a decreased patient survival, preferably a shorter progression-free survival.

The present invention also concerns a method for selecting a subject affected with a non-muscle invasive bladder cancer with an activating mutation in FGFR3 gene for an anti-tumoral therapy, or for determining whether a subject affected with a non-muscle invasive bladder cancer with an activating mutation in FGFR3 gene is susceptible to benefit from an anti-tumoral therapy, wherein the method comprises detecting a loss-of-function mutation in CDKN2A gene in bladder cancer cells from the subject, the presence of a loss-of-function mutation in the CDKN2A gene being indicative that an anti-tumoral therapy is required for the subject affected with the non-muscle invasive bladder cancer with an activating mutation in FGFR3 gene. The anti-tumoral therapy may be selected from the group consisting of adjuvant or neoadjuvant therapy, immunotherapy, preferably BCG therapy, and/or partial or radical cystectomy.

The present invention further concerns a method for providing information for determining follow-up strategy of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative that an intensive follow-up is required for the subject affected with the non-muscle invasive bladder cancer with an activating mutation in FGFR3 gene.

Preferably, the loss-of-function mutation in CDKN2A gene is a hemizygous or homozygous deletion of the CDKN2A gene.

Preferably, the activating mutation in FGFR3 gene is selected from the group consisting of R248C, S249C, P250R, G372C, S373C, Y375C, G382R, A391E, K652E, K652Q, K652M and K652T, and a combination thereof.

The methods of the invention may further comprise detecting a hemizygous or homozygous deletion at chromosome 11p region and/or may further comprise assessing at least one other cancer or prognosis markers such as tumor stage, grade, number of tumors, prior recurrence rate mitotic index, tumor size, HJURP expression level, HP1α expression level or expression of proliferation markers such as Ki67, MCM2, CAF-1 p60 and CAF-1 p150.

In another aspect, the present invention concerns a kit (a) for predicting or monitoring clinical outcome of a subject affected with a non-muscle invasive bladder cancer; and/or (b) for selecting a subject affected with a non-muscle invasive bladder cancer for an anti-tumoral therapy, or for determining whether a subject affected with a non-muscle invasive bladder cancer is susceptible to benefit from an anti-tumoral therapy; and/or (c) for providing information for determining follow-up strategy of a subject affected with a non-muscle invasive bladder cancer, wherein the kit comprises (i) at least one probe specific to CDKN2A gene and/or at least one nucleic acid primer pair specific to CDKN2A gene, and (ii) at least one probe specific to FGFR3 gene and/or at least one nucleic acid primer pair specific to FGFR3 gene, and optionally, a leaflet providing guidelines to use such a kit.

The kit may comprise at least 5, 10, 15, 20, 25, 30, 40 or 50 probes or primers specific to CDKN2A gene. Preferably, the kit comprises at least one probe or primer specific to a CDKN2A sequence selected from the sequences of SEQ ID NOs: 1 to 15. More preferably, the kit comprises at least 10 probes selected from the group consisting of probes specific to a CDKN2A sequence selected from the sequences of SEQ ID NOs: 1 to 15.

The kit may comprise at least one probe or primer specific to FGFR3 gene suitable to detect at least one FGFR3 mutation selected from the group consisting of R248C, S249C, P250R, G372C, S373C, Y375C, G382R, A391E, K652E, K652Q, K652M and K652T, and any combination thereof.

The kit may also comprise at least one probe specific to chromosome 11p region and/or at least one nucleic acid primer pair specific to chromosome 11p region.

Preferably, the kit further comprise at least one reference probe specific to a chromosomal region that is rarely altered in bladder cancer and/or at least one nucleic acid primer pair specific to a chromosomal region that is rarely altered in bladder cancer.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1C: CDKN2A deletions as a function of stage in FGFR3-mutated tumors and in FGFR3-wild-type tumors. FIG. 1A. The percentage of tumors displaying homozygous CDKN2A deletion (in white), hemizygous deletion (light grey) and not deleted (dark grey) is shown as a function of stage in FGFR3-mutated samples (upper panel) and FGFR3-wild-type samples (lower panel). FIG. 1B. Association of CDKN2A homozygous deletions with the stage in FGFR3-mutated tumors (upper panel) and in FGFR3-wild-type tumors (lower panel). Are shown the number of tumors in each category and in parentheses the percentage of tumors of each category within a given stage. These different percentages are also represented in A. no D, absence of CDKN2A deletion; hD, CDKN2A hemizygous deletion; HD, CDKN2A homozygous deletion. *P-values were calculated by comparing the number of CDKN2A homozygous deletions to the number of no deletion+hemizygous deletions, using two-tailed Fisher's exact tests. FIG. 1C. Association of CDKN2A homozygous deletions with the FGFR3-mutation status at each stage. Are shown the number of tumors in each category and in parentheses the percentage of tumors of each category within a given stage. These different percentages are also represented in A. no D, absence of CDKN2A deletion; hD, CDKN2A hemizygous deletion; HD, CDKN2A homozygous deletion. *P-values were calculated by comparing the number of CDKN2A homozygous deletions to the number of no deletion+hemizygous deletions, using two-tailed Fisher's exact tests.

FIGS. 2A-2C: CDKN2A deletions as a function of grade in FGFR3-mutated tumors and in FGFR3-wild-type tumors. FIG. 2A. The percentage of tumors displaying homozygous CDKN2A deletion (in white), hemizygous deletion (light grey) and not deleted (dark grey) is shown as a function of grade in FGFR3-mutated samples (upper panel) and FGFR3-wild-type samples (lower panel). FIG. 2B. Association of CDKN2A homozygous deletions with the grade in FGFR3-mutated tumors (upper panel) and in FGFR3-wild-type tumors (lower panel). Are shown the number of tumors in each category and in parentheses the percentage of tumors of each category within a given grade. These different percentages are also represented in A. no D, absence of CDKN2A deletion; hD, CDKN2A hemizygous deletion; HD, CDKN2A homozygous deletion. *P-values were calculated by comparing the number of CDKN2A homozygous deletions to the number of no deletion+hemizygous deletions, using two-tailed Fisher's exact tests. FIG. 2C. Association of CDKN2A homozygous deletions with the FGFR3-mutation status at each grade. Are shown the number of tumors in each category and in parentheses the percentage of tumors of each category within a given grade. These different percentages are also represented in A. no D, absence of CDKN2A deletion; hD, CDKN2A hemizygous deletion; HD, CDKN2A homozygous deletion. *P-values were calculated by comparing the number of CDKN2A homozygous deletions to the number of no deletion+hemizygous deletions, using two-tailed Fisher's exact tests.

FIGS. 3A-3B: Deletion of CDKN2A is associated with progression in non-muscle-invasive FGFR3-mutated tumors. FIG. 3A. Kaplan-Meier analysis of recurrence and invasive progression in a cohort of 89 patients (maximum follow up=11.7 years, median=3.5) after the resection of a non-muscle-invasive bladder tumor with FGFR3 mutation. In the left panels, three groups of tumors have been compared: no deletion (CDKN2A+/+), hemizygous deletion (CDKN2A+/−), or homozygous deletion of the CDKN2A locus (CDKN2A−/−). In the right panels, two groups of tumors have been compared: no deletion (CDKN2A+/+), hemizygous deletion and homozygous deletion of the CDKN2A locus (CDKN2A+/−, −/−). P-values were determined in log rank tests. FIG. 3B. Fluorescence in situ hybridization (FISH) of the primary non muscle-invasive tumor from P159 and its invasive progression. The centromere of chromosome 9 is labeled green and the CDKN2A locus is labeled red. Single copies of both loci are present in the non muscle-invasive tumor, but CDKN2A, but not the centromeric probe, has been entirely lost from the invasive sample. White arrow indicates a lymphocyte.

FIG. 4: FGFR3 mutation and CDKN2A deletion as a function of stage and grade in the series of 288 bladder tumors.

### DETAILED DESCRIPTION OF THE INVENTION

In bladder cancers, the cyclin-dependent kinase inhibitor 2A gene (CDKN2A, Gene ID: 1029) is frequently inactivated by deletion. However the role of this gene in bladder tumorigenesis remains unclear. The CDKN2A gene, located at 9p21, encodes two tumor suppressor proteins of unrelated amino-acid sequences, p16^{INK4a} and p14^{ARF}. p16^{INK4a} is involved in cell cycle arrest and induction of senescence. It inhibits cyclin-dependent kinases and therefore maintains the retinoblastoma gene product (Rb) in its hypophosphorylated active state. p14^{ARF} is involved in the p53 pathway stabilizing p53 through inhibition of MDM2. CDKN2A is altered in many different cancers by different mechanisms. In bladder tumors, while mutation and promoter hypermethylation are rare, hemizygous and homozygous deletions are common, being found in 40-60% and in 10-30% of cases, respectively (Berggren et al., 2003; Chapman et al., 2005; Florl et al., 2000; Orlow et al., 1995; Orlow et al., 1999; Williamson et al., 1995). Hemizygous deletions usually span the whole of chromosome arm 9p and therefore cannot be directly associated with CDKN2A. By contrast, homozygous deletions are usually restricted to CDKN2A, sometimes including the neighboring centromeric gene, CDKN2B, another tumor suppressor gene which codes for p15^{INK4b}, a cell cycle inhibitor. The association of CDKN2A deletions with clinical and pathologic parameters has been evaluated in several studies, giving conflicting findings. Some studies reported no association with stage/grade (Berggren et al., 2003; Florl et al., 2000), whereas others suggested an association with low-stage/grade tumors (Orlow et al., 1995), with recurrence (Orlow et al., 1999) or with recurrence and invasion (Chapman et al., 2005). None of these studies has evaluated the CDKN2A deletion status with regards to the two pathways of bladder tumor progression.

The inventors have investigated the frequency of CDKN2A deletions according to the FGFR3 mutation status in a series of 288 bladder tumors. They have also examined if FGFR3-mutated non-muscle-invasive bladder tumors displaying a loss of one or two copies of the CDKN2A gene would be more prone to progress than FGFR3-mutated non-muscle-invasive tumors without deletion of CDKN2A. They have thus herein demonstrated that CDKN2A losses (hemizygous or homozygous losses) are associated with progression of non-invasive FGFR3-mutated bladder tumors, and thus with shorter progression free survival, whereas there is no significant association between CDKN2A losses and stage or grade of FGFR3-wildtype bladder tumors.

Accordingly, in a first aspect, the present invention concerns a method for predicting clinical outcome of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative of a non-muscle invasive bladder cancer with FGFR3 activating mutation with a poor prognosis.

The methods of the invention as disclosed herein, may be in vivo, ex vivo or in vitro methods, preferably in vitro methods.

By “bladder tumor” or “bladder cancer” is intended herein urinary bladder tumor, urinary bladder cancer, and bladder neoplasm or urinary bladder neoplasm. The most common staging system for bladder tumors is the TNM (tumor, node, metastasis) system (Sobin et al., 1997). This staging system takes into account how deep the tumor has grown into the bladder, whether there is cancer in the lymph nodes and whether the cancer has spread to any other part of the body. The TNM classification comprises the following stages:

Ta—the cancer is just in the innermost layer of the bladder lining;

T1—the cancer has started to grow into the connective tissue beneath the bladder lining;

T2—the cancer has grown through the connective tissue into the muscle;

T2a—the cancer has grown into the superficial muscle;

T2b—the cancer has grown into the deeper muscle;

T3—the cancer has grown through the muscle into the fat layer;

T3a—the cancer in the fat layer can only be seen under a microscope;

T3b—the cancer in the fat layer can be seen on tests, or felt by the physician;

T4—the cancer has spread outside the bladder;

T4a—the cancer has spread to the prostate, womb or vagina;

T4b—the cancer has spread to the wall of the pelvis and abdomen.

The term “non-muscle-invasive bladder cancer” or “non-muscle-invasive bladder tumor”, as used herein, thus refers to Ta or T1 bladder tumor.

The term “bladder tumor with an activating mutation in FGFR3 gene”, as used herein, refers to bladder tumor comprising cells harbouring at least one mutation of the FGFR3 (fibroblast growth factor receptor 3) gene leading to constitutive activation of the receptor (see for example the international patent application WO 00/68424). The FGFR3 gene (Gene ID: 2261) is located at 4p16.3. Three alternatively spliced transcript variants that encode different protein isoforms have been described. In a preferred embodiment, the FGFR3 mutation is selected from the group consisting of R248C, S249C, P250R, G372C, S373C, Y375C, G382R, A391E, K652E, K652Q, K652M and K652T, and a combination thereof. Codons are numbered according to FGFR3b cDNA ORF (Cappellen et al., 1999).

As used herein, the term “poor prognosis” refers to a shorter progression-free survival and/or an increased metastasis occurrence and/or a decreased patient survival. Preferably, this term refers to a shorter progression-free survival.

As used herein, the term “subject” or “patient” refers to an animal, preferably to a mammal, even more preferably to a human, including adult, child and human at the prenatal stage.

The method may further comprise the step of providing a biological sample comprising bladder cancer cells from said subject.

Bladder cancer cells may be obtained from any biological sample from the subject comprising such cells. In particular, Bladder cancer cells may be obtained from fluid sample such as blood, plasma, urine and seminal fluid samples as well as from biopsies, organs, tissues or cell samples. In a preferred embodiment, bladder cancer cells are obtained from urine sample or bladder tumor biopsy sample from the subject. Samples containing bladder cancer cells may be treated prior to its use. As example, a tumor cell enrichment sorting may be performed.

The method of the invention comprises detecting a loss of function mutation in CDKN2A gene in bladder cancer cells.

As used herein, the term “mutation” encompasses point mutation, deletion, rearrangement and/or insertion in the coding and/or non-coding region of the locus, alone or in various combination(s). Deletions may encompass any region of one, two or more residues in a coding or non-coding portion of the gene locus, such as from two residues up to the entire gene or locus. Insertions may encompass the addition of one or several residues in a coding or non-coding portion of the gene locus. Insertions may typically comprise an addition of between 1 and 50 base pairs in the gene locus. Rearrangement includes inversion of sequences. The mutation may result in the creation of stop codons, frameshift mutations, amino acid substitutions, altered or prevented RNA splicing or processing, product instability, truncated polypeptide production, etc. The mutation may result in the production of a polypeptide with altered function, stability, targeting or structure. It may also cause a reduction or a complete inhibition of protein expression.

The term “loss-of-function mutation” refers to a mutation which affects the activity of the protein(s) encoded by the mutated gene. The protein(s) may be less active or completely inactive, preferably completely inactive. As used herein, the term “loss-of-function mutation in CDKN2A gene” refers to a mutation which affects the activity of the protein p16^{INK4a} and/or of the protein p14^{ARF}. Preferably, the mutation affects the activity or expression of these two proteins. More preferably, the mutation completely inhibits the activity or expression of these two proteins.

In a preferred embodiment, the loss-of-function mutation in CDKN2A gene is a deletion of all or part of CDKN2A gene. Preferably, the deletion is more than 25, 50, 100, 200, 300, 500, 1000, 2000, 5000 or 10000 nucleotides in length. Preferably, the deletion involves the CDKN2A coding sequence(s).

In a particular embodiment, the loss-of-function mutation in CDKN2A gene is the complete deletion of the CDKN2A gene. The deletion may also include the centromeric neighboring gene CDKN2B encoding P15^{INK4b}, another tumor suppressor, and/or the neighboring telomeric gene MTAP.

The loss-of-function mutation in CDKN2A gene can be detected by any method known by the skilled person. This mutation can be detected at the DNA, RNA or protein level.

In a first embodiment, the loss-of-function mutation in CDKN2A gene is detected at the DNA level, for instance by sequencing all or part of CDKN2A gene, using selective hybridization and/or amplification of all or part of CDKN2A gene, or restriction digestion.

In particular, a deletion of all or part of CDKN2A gene can be detected by various methods known in the art such as, for example, Multiplex Ligation-dependent Probe Amplification (MLPA), FISH, oligonucleotide-based array CGH or Nanostring technology. Oligonucleotide probes used in these methods can be easily designed by the person skilled in the art. In a preferred embodiment, the deletion of all or part of CDKN2A gene is detected by MLPA method. MLPA kits specific of the CDKN2A/2B region are commercially available (e.g. SALSA MLPA kit ME024-B19p21 CDKN2A/2B region, MRC-Holland, Amsterdam, The Netherlands).

In another embodiment, the loss-of-function mutation in CDKN2A gene is detected at the RNA level, for instance, by detecting a RNA mutated sequence or an abnormal RNA splicing or processing or expression level. This detection may be carried out by various techniques known in the art, including restriction digestion, sequencing of all or part of the RNA of interest, selective hybridization or selective amplification of all or part of said RNA. In particular, the mutation may be detected using northern-blot or quantitative RT-PCR.

In a further embodiment, the loss-of-function mutation in CDKN2A gene is detected at the protein level, for instance by detecting a mutated polypeptide sequence or an impaired expression of the protein P16^{INK4a} and/or P14^{ARF}.

A mutated polypeptide sequence can be detected by various techniques known in the art such as, for example, polypeptide sequencing and/or binding to specific ligands such as antibodies. An impaired or an absence of expression of P16^{INK4a} and/or P14^{ARF} protein can also be detected by various techniques known in the art such as, for instance, Western-blot, ELISA, radio-immunoassay or immuno-enzymatic assays. Polyclonal and monoclonal antibodies directed against P16^{INK4a} or P14^{ARF} are commercially available. Examples of anti-P16^{INK4a} marketed antibodies are p16 C20 polyclonal antibody (Santa Cruz Biotechnology, Santa Cruz, Calif.), anti-p16^{INK4a} antibody (clone 16P07) (Neomarkers). Examples of anti-P14^{ARF} marketed antibodies are monoclonal anti-P14^{ARF} antibody (clone 14P03) (Neomarkers) and rabbit polyclonal anti-p14^{ARF} antibody (Neomarkers).

Genomic DNA extraction and purification, restriction digestions, sequencing reactions, selective hybridization and selective amplification may be carried out according to well-known protocols such as those described in Sambrook et al. (Sambrook et al. Molecular Cloning, A Laboratory Manual, Third Edition, Cold Spring Harbor Laboratory Press, 2000) and in Ausubel et al. (In Current Protocols in Molecular Biology, John Wiley & Sons, NY, 1998).

In an embodiment, the loss-of-function mutation affects only one copy of the CDKN2A gene (hemizygous mutation). In another embodiment, the loss-of-function mutation affects the two copies of the CDKN2A gene (homozygous mutation). In this embodiment, the mutations in each copy can be identical or different.

In a particular embodiment, the loss-of-function mutation in CDKN2A gene is a hemizygous or homozygous deletion of the CDKN2A gene, preferably a homozygous deletion.

The inventors also found that deletions at chromosome 11p region are correlated with stage in bladder tumors. Accordingly, in an embodiment, the method of the invention further comprises detecting a hemizygous or homozygous deletion at chromosome 11p region. Deletion of this region may be detected using any method known by the skilled person such as methods as described above.

The method may further comprise assessing at least one other cancer or prognosis markers such as tumor stage, grade, number of tumors, prior recurrence rate, mitotic index, tumor size, HJURP expression level, HP1α expression level (WO 2010/122137) or expression of proliferation markers such as Ki67, MCM2, CAF-1 p60 and CAF-1 p150. These markers are commonly used and the results obtained with these markers may be combined with the results obtained with the present method in order to confirm the prognosis. The use of these markers is well-known by the skilled person. As example, the tumor grade may be determined according the method disclosed in Mostofi et al., 1973, the mitotic index may be determined by counting mitotic cells in ten microscopic fields of a representative tissue section and the tumor size can be detected by imaging techniques, by palpation or after surgery in the excised tissue. Expression of Ki67 and CAF-1 can be assessed at the protein level or at the mRNA level. High grade, high mitotic index, large size and/or high Ki67 or CAF-1 expression are indicative for a worse prognosis. The HJURP expression level may be assessed as described in Hu et al., 2010. High HJURP expression is associated with poor clinical outcomes. In a particular embodiment, the method of the invention further comprise determining the EORTC (European Organization for Research and Treatment of Cancer) risk score as disclosed in the article of Sylvester et al., 2006.

In second aspect, the present invention concerns a method for selecting a subject affected with a non-muscle invasive bladder cancer with an activating mutation in FGFR3 gene for an anti-tumoral therapy, or for determining whether a subject affected with a non-muscle invasive bladder cancer with an activating mutation in FGFR3 gene is susceptible to benefit from an anti-tumoral therapy, wherein the method comprises detecting a loss-of-function mutation in CDKN2A gene in cancer cells from the subject, the presence of a loss-of-function mutation in the CDKN2A gene being indicative that an anti-tumoral therapy is required for the subject affected with the non-muscle invasive bladder cancer with an activating mutation in FGFR3 gene.

All the embodiments of the method for predicting clinical outcome as described above are also contemplated in this method.

As used herein, the term “anti-tumoral therapy” refers to any act intended to ameliorate the health status of patients affected with a cancer such as therapy, prevention and retardation of the disease. In certain embodiments, this term refers to a therapy inducing amelioration or eradication of the cancer or symptoms associated with said cancer. In other embodiments, this term refers to a therapy minimizing the spread or worsening of the cancer.

The anti-tumoral therapy may be chemotherapy, radiotherapy, immunotherapy, surgery and/or hormone therapy. Preferably, the anti-tumoral therapy is chemotherapy, radiotherapy, immunotherapy and/or surgery.

Surgery for bladder cancer may include transurethral resection of the bladder, i.e. cancerous bladder tissue is removed through the urethra, and partial or complete removal of the bladder (radical cystectomy).

As used herein, the term “chemotherapy” refers to a cancer therapeutic treatment using chemical or biochemical substances, in particular using one or several antineoplastic agents. For treating non invasive bladder cancer, chemotherapy is usually given directly into the bladder. The chemotherapy may involve administration of cisplatin, adriamycin, mitomycin C, gemcitabine, paclitaxel, docetaxel tamoxifen, aromatase inhibitors, trastuzumab, GnRH-analogues, carboplatin, oxaliplatin, doxorubicin, daunorubicin cyclophosphamide, epirubicin, fluorouracil, methotrexate, mitozantrone, vinblastine, vincristine, vinorelbine, bleomycin, estramustine phosphate or etoposide phosphate, or any combination thereof. Preferably, the chemotherapy involves the administration of an antineoplastic agent selected from the group consisting of cisplatin, adriamycin, mitomycin C, gemcitabine, paclitaxel, doxorubicin or docetaxel, or any combination thereof.

Chemotherapy frequently causes vomiting, nausea, alopecia, mucositis, myelosuppression particularly neutropenia, sometimes resulting in septicaemia. Depending on the antineoplastic agent used, chemotherapy can also cause bladder damage, constipation or diarrhea, neuropathy, hemorrhage, leukoencephalopathy or post-chemotherapy cognitive impairment.

The term “radiotherapy” is a term commonly used in the art to refer to multiple types of radiation therapy including internal and external radiation therapy, radioimmunotherapy, and the use of various types of radiation including X-rays, gamma rays, alpha particles, beta particles, photons, electrons, neutrons, radioisotopes, and other forms of ionizing radiation.

Radiotherapy can cause radiation dermatitis, fatigue, nausea, vomiting, diarrhea, prostitis, proctitis, dysuria, metritis and abdominal pain.

As used herein, the term “immunotherapy” refers to a medication that triggers the immune system of the subject to attack and kill the cancer cells. Immunotherapy for bladder cancer may be performed using the Bacille Calmette-Guerin vaccine (commonly known as BCG) directly administered into the bladder (intravesical administration). Immunotherapy may also involve interferon administration, usually when BCG treatment does not work. Intravesical BCG, optionally in combination with transurethral resection of bladder tumor, is an effective treatment for non-muscle invasive bladder cancer. BCG therapy has been shown to delay tumor progression, decrease the need for surgical removal of the bladder at a later time, and improve overall survival. However, most people who are treated with intravesical BCG have some side effects. The most common of these include the need to urinate frequently, pain with urination, fever, blood in the urine, and body aches. However, less common but more serious side effects can also appear such as body wide infection.

Radiotherapy, chemotherapy or immunotherapy may be given as adjuvant treatment after surgical resection of the primary bladder tumor in a patient affected with a cancer that is at risk of progressing and/or metastasizing. The aim of such an adjuvant treatment is to improve the prognosis. Radiotherapy, chemotherapy or immunotherapy may also be given as neoadjuvant treatment before surgery.

The detection of a loss-of-function mutation in CDKN2A gene in cancer cells from the subject indicates a shorter progression free survival and/or an increased metastasis occurrence and/or a decreased patient survival. Accordingly, this type of bladder cancer associated with poor prognosis has to be treated with adjuvant or neoadjuvant chemotherapy, radiotherapy, immunotherapy in order to improve the patient's chance for survival. The type of adjuvant or neoadjuvant therapy is chosen by the practitioner. Furthermore, partial or radical cystectomy, preferably radical cystectomy, may be advised for patients affected with a bladder cancer with poor prognosis. Due to prejudicial side effect of the antitumoral treatment, it is helpful to discriminate and select the patients who really need such a treatment.

In a further aspect, the present invention also concerns a method for providing information for determining follow-up strategy of a subject affected with a non-muscle invasive bladder cancer with an activating mutation in FGFR3 gene, wherein the method comprises a step of detecting a loss-of-function mutation in CDKN2A gene in cancer cells from the subject, the presence of a loss-of-function mutation in the CDKN2A gene being indicative that an intensive follow-up is required for the subject affected with the non-muscle invasive bladder cancer with an activating mutation in FGFR3 gene.

All the embodiments of the method for predicting clinical outcome as described above are also contemplated in this method.

The detection of a loss-of-function mutation in CDKN2A gene in cancer cells from the subject indicates a shorter progression free survival. Accordingly, for patients affected with bladder cancer with poor prognosis, an intensive follow up after treatment is required to monitor for recurrence and progression. Surveillance can include cystoscopy, urine cytology testing, CT scans, complete blood count, monitoring symptoms that might suggest the recurrence or progression of disease, such as fatigue, weight loss, increased pain, decreased bowel and bladder function, and weakness.

Usually, the follow-up after treatment of bladder cancer includes at least cystoscopy and urine cytology testing every three to six months for four years, and then once per year. For patient affected with a non invasive FGFR3-mutated bladder cancer with a loss-of-function mutation in CDKN2A gene, the practitioner may advise a more intensive follow-up in order to rapidly detect any progression of the disease.

The present invention also concerns
(a) a method for predicting clinical outcome of a subject affected with a non-muscle invasive bladder cancer, wherein the method comprises (i) detecting an activating mutation in FGFR3 gene in bladder cancer cells from the subject; (ii) detecting a loss-of-function mutation in CDKN2A gene in bladder cancer cells from the subject, the presence of an activating mutation in FGFR3 gene and a loss-of-function mutation in the CDKN2A gene being indicative of a non-muscle invasive bladder cancer with a poor prognosis;
(b) a method for selecting a subject affected with a non-muscle invasive bladder cancer for an anti-tumoral therapy, or determining whether a subject affected with a non-muscle invasive bladder cancer is susceptible to benefit from an anti-tumoral therapy, wherein the method comprises (i) detecting an activating mutation in FGFR3 gene in bladder cancer cells from the subject; (ii) detecting a loss-of-function mutation in CDKN2A gene in bladder cancer cells from the subject, the presence of an activating mutation in FGFR3 gene and a loss-of-function mutation in the CDKN2A gene being indicative that an anti-tumoral therapy is required for the subject affected with the non-muscle invasive bladder cancer; and
(c) a method for providing information for determining follow-up strategy of a subject affected with a non-muscle invasive bladder cancer, wherein the method comprises (i) detecting an activating mutation in FGFR3 gene in bladder cancer cells from the subject; (ii) detecting a loss-of-function mutation in CDKN2A gene in bladder cancer cells from the subject, the presence of an activating mutation in FGFR3 gene and a loss-of-function mutation in the CDKN2A gene being indicative that an intensive follow-up is required for the subject affected with the non-muscle invasive bladder cancer. All the embodiments of the methods as described above are also contemplated in this aspect.

In an embodiment, the FGFR3 mutation is selected from the group consisting of R248C, S249C, P250R, G372C, S373C, Y375C, G382R, A391E, K652E, K652Q, K652M and K652T, and a combination thereof. Activating mutations in FGFR3 gene can be detected by any method known by the skilled person. FGFR3 mutations can be detected for example by sequencing all or part of FGFR3 gene or by the SNaPshot method as described in the article of van Oers et al., 2005 or by the method disclosed in the article of Bakkar et al., 2005.

Optionally, if in step (i), the presence of an activating mutation of FGFR3 is not detected, then step (ii) can be omitted.

The present invention also concerns a kit (a) for predicting or monitoring clinical outcome of a subject affected with a non-muscle invasive bladder cancer; and/or (b) for selecting a subject affected with a non-muscle invasive bladder cancer for an anti-tumoral therapy, or for determining whether a subject affected with a non-muscle invasive bladder cancer is susceptible to benefit from an anti-tumoral therapy; and/or (c) for providing information for determining follow-up strategy of a subject affected with a non-muscle invasive bladder cancer, wherein the kit comprises (i) at least one probe specific to CDKN2A gene and/or at least one nucleic acid primer pair specific to CDKN2A gene, and (ii) at least one probe specific to FGFR3 gene and/or at least one nucleic acid primer pair specific to FGFR3 gene, and optionally, a leaflet providing guidelines to use such a kit.

Oligonucleotide probes or primers specific to CDKN2A or FGFR3 gene can be easily designed by the person skilled in the art.

In an embodiment, the kit comprises at least 5, 10, 15, 20, 25, 30, 40 or 50 probes or primers specific to CDKN2A gene. Examples of probes specific to CDKN2A gene include, but are not limited to, probes specific of a CDKN2A sequence selected from the sequences of SEQ ID NOs: 1 to 15. Preferably, the kit comprises at least 10 probes selected from the group consisting of probes specific of a CDKN2A sequence selected from the sequences of SEQ ID NOs: 1 to 15.

In an embodiment, the kit comprises probes or primers specific to FGFR3 gene suitable to detect at least one FGFR3 mutation selected from the group consisting of R248C, S249C, P250R, G372C, S373C, Y375C, G382R, A391E, K652E, K652Q, K652M and K652T, and any combination thereof.

In another embodiment, the kit further comprises at least one probe specific to chromosome 11p region and/or at least one nucleic acid primer pair specific to chromosome 11p region. Preferably, the probe or the nucleic acid primer is specific to a region extending from the chromosomal location 11p13 to the 11p telomere.

In a further embodiment, the kit further comprises at least one, preferably several, reference probe(s) specific to a chromosomal region that is rarely altered in bladder cancer and/or at least one, preferably several, nucleic acid primer pair(s) specific to a chromosomal region that is rarely altered in bladder cancer. This reference probe or primer may be used as internal control during mutation analysis. In a particular embodiment, the reference probe or primer is specific to a chromosomal location selected from the group consisting of 2q14, 5q31, 1p22, 7p22, 14q24, 5q35, 11q13, 17p13, 8q24, 5q35, 7q11, 22q11, 2p14, 22q13 and 10p14. In another particular embodiment, the reference probe or primer is specific to a chromosomal region selected from the group consisting of the region from 1p34.2 to 1p12.1, the region from tel2 to 2p11.1, the chromosome 12 except the region comprising 10 Mb downstream and 10 Mb upstream the MDM2 gene, and the chromosome 21.

The present invention also concerns a use of a kit comprising (i) at least one probe specific to CDKN2A gene and/or at least one nucleic acid primer pair specific to CDKN2A gene, and (ii) at least one probe specific to FGFR3 gene and/or at least one nucleic acid primer pair specific to FGFR3 gene, and optionally, a leaflet providing guidelines to use such a kit,

(a) for predicting or monitoring clinical outcome of a subject affected with a non-muscle invasive bladder cancer; and/or

(b) for selecting a subject affected with a non-muscle invasive bladder cancer for an anti-tumoral therapy, or determining whether a subject affected with a non-muscle invasive bladder cancer is susceptible to benefit from an anti-tumoral therapy; and/or

(c) for providing information for determining follow-up strategy of a subject affected with a non-muscle invasive bladder cancer.

All the embodiments of the kit as described above are also contemplated in this use.

The present invention also concerns a use of a kit comprising at least one probe specific to CDKN2A gene and/or at least one nucleic acid primer pair specific to CDKN2A gene, and optionally, a leaflet providing guidelines to use such a kit,

(a) for predicting or monitoring clinical outcome of a subject affected with a non-muscle invasive bladder cancer with an activating mutation in FGFR3 gene; and/or

(b) for selecting a subject affected with a non-muscle invasive bladder cancer with an activating mutation in FGFR3 gene for an anti-tumoral therapy, or determining whether a subject affected with a non-muscle invasive bladder cancer with an activating mutation in FGFR3 gene is susceptible to benefit from an anti-tumoral therapy; and/or

(c) for providing information for determining follow-up strategy of a subject affected with a non-muscle invasive bladder cancer with an activating mutation in FGFR3 gene.

In an embodiment, the kit further comprises at least one probe specific to FGFR3 gene and/or at least one nucleic acid primer pair specific to FGFR3 gene.

All the embodiments of the kit as described above are also contemplated in this use.

Further aspects and advantages of the present invention will be described in the following examples, which should be regarded as illustrative and not limiting.

### EXAMPLES

Materials and Methods

Patients

Two hundred and eighty eight bladder tumor samples were obtained from 288 patients. These patients were included prospectively between 1988 and 2006 at Henri Mondor and Foch hospitals, and in the framework of an epidemiological case-control study for Necker Hospital and Institut Mutualiste Montsouris for molecular studies (Michiels et al., 2009). Tumors were staged according to the 1997 TNM classification and grades were classified according to the 1973 WHO classification (Mostofi et al., 1973). The 288 tumors comprised 177 non-muscle-invasive tumors (123 Ta, 54 T1) and 111 muscle-invasive tumors (35 T2, 46 T3 and 30 T4). The grade distribution was 44 grade 1 (G1), 92 grade 2 (G2) and 152 grade 3 (G3). Of the 177 patients with a non-muscle-invasive tumor, 151 were new cases, 17 were recurrent cases, and the status was not available for 9 cases (Table 1). Of the 111 patients with a muscle-invasive tumor, 86 were new cases, 4 were recurrent cases, 19 were progression cases and the status was not available for 2 cases (Table 1).

All subjects provided informed consent and the study was approved by the ethics committees of the different hospitals.

DNA Extraction

Flash-frozen tumor samples were stored at −80° C. immediately after transurethral resection or cystectomy. All tumor samples contained >80% tumor cells, as assessed by hematoxylin and eosin staining of histological sections adjacent to the samples used for genome analysis. Tumor samples were disrupted by thawing and blending with an Ultraturax, and DNA was extracted by cesium chloride density centrifugation (Coombs et al., 1990). DNA purity was assessed by determining the ratio of absorbances at 260 and 280 nm. DNA concentration was determined with a Hoechst dye-based fluorescence assay (Labarca et al., 1980).

Fibroblast Growth Factor Receptor 3 Mutation Analysis

FGFR3 mutations were assessed by the SNaPshot method as previously described (Van Oers et al., 2005). The SNaPshot method is based on the dideoxy single-base extension of unlabeled oligonucleotide primers. Briefly, exons 7, 10 and 15 were analyzed for mutations using the ABI PRISM SNaPshot Multiplex Kit (Applied Biosystems, Foster City, Calif.), according to the protocol supplied by the manufacturer. The data were analyzed with GeneScan Analysis Software version 3.7 (Applied Biosystems). The primers used allowed the detection of the following codon mutations: R248C and S249C (exon 7), G372C, Y375C, and A391E (exon 10), and K652E, K652Q, K652M and K652T (exon 15). These mutations account for more than 99% of the FGFR3 mutations found in bladder carcinomas (Van Rhijn et al., 2002).

Multiplex Ligation-Dependent Probe Amplification (MLPA)

MLPA was carried out as described elsewhere (Aveyard et al., 2004). Briefly, 50 ng of bladder tumor DNA was analyzed with the P024B kit (MRC-Holland, Amsterdam, The Netherlands), containing 23 probes covering the CDKN2A/2B genes and surrounding regions and 17 control probes targeting other chromosomes. Probe hybridization, ligation, and PCR were carried out according to the manufacturer's protocol. Dosage quotients were calculated as described by MRC-Holland, using peak heights rather than peak areas. Peak heights were normalized to 2 in two steps. First, control probes were used to adjust normal levels across samples. The peak height of each probe was then normalized against the peak heights of the same probe in normal samples. This second step corrects for differences in efficiency between probes. It was considered that peak heights above 1.6 to indicate the absence of deletion (normal copy number or gain), peak heights between 1.6 and 0.8 to indicate hemizygous deletion and peak heights below 0.8 to indicate homozygous deletion.

Fluorescence In Situ Hybridization (FISH)

For some selected samples, CDKN2A was also assessed by Dual-color FISH. Dual-color FISH analysis was performed using the Vysis LSI p16 (9p21) Spectrum Orange and CEP-9 Spectrum Green probes (Abbot molecular, Wiesbaden, Germany). In brief, paraffin sections were de-waxed, re-hydrated, washed and pre-treated using Histology FISH Accessory Kit (Dako, Glostrup, Denmark), with an enzymatic digestion with pepsin at 37° C. for 15 mn. The slides were co-denatured at 85° C. for 1 mn, hybridized at 37° C. for 20 hrs (Hybridizer, Dako Cytomation), and washed in 2×SCC/0.3% Tween20 at 73° C. for 2 mn. Nuclei were counterstained with DAPI/antifade. Images were captured with 40× objective using a Hamamatsu digital camera attached to the fluorescence microscope and Pathfinder software (IMSTAR, Paris, France). The histological areas previously selected on the H&E-stained sections were identified on the FISH-treated slides. Control tissue included normal urothelium and bladder muscle. At least 100 cells were scored for each case and control. Each tumor was assessed by determining the mean number of copies of the CDKN2A gene per cell and the mean ratio of CDKN2A gene copy number to chromosome 9 copy number (CEP-9). Homozygous deletion was considered to have occurred if both 9p21 signals were lost in at least 20% of nuclei with at least one signal for the CEP-9 probe.

Statistical Analyses

Association of CDKN2A copy number with stage or grade was assessed through a chi-squared test for trend in proportions with scores set to 0 for stage Ta (or G1 for grade-based analyses), 1 for T1 (or G2) and 2 for T2-4 (or G3) tumors. A two-tailed Fisher's exact test was used to assess association with FGFR3 mutation or enrichment at a particular stage.

Follow-Up Analyses of Non-Muscle-Invasive FGFR3 Mutated Tumors

Follow-up analyses were available for 89 patients with non-muscle-invasive FGFR3-mutated tumors. The maximum follow-up duration was 11.7 years and the median follow-up was 3.5 years. Kaplan-Meier analysis of recurrence and progression was carried out using GraphPad Prism version 5 software. Recurrence was defined as the appearance of a new tumor of any stage, whereas progression was restricted to any of the following: appearance of a muscle-invasive tumor, metastasis or cancer-specific death. Data were censored after the last follow-up consultation had occurred or after patients died from a cause other than cancer. The log-rank test was used to assess the significance of the differences observed.

Results

FGFR3 Mutations and CDKN2A Deletions

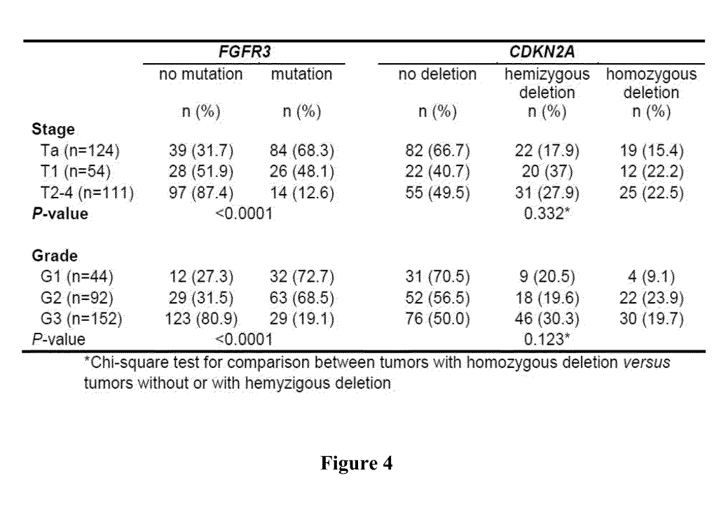

FGFR3 mutation status and the DNA copy number at the CDKN2A locus for the 288 tumors are given in Table 1. One hundred and twenty four FGFR3-mutated samples (43.1%) and 56 samples with CDKN2A homozygous deletions (19.4%) were identified. The distribution of these alterations as a function of stage and grade is given in Table 1. As already reported in numerous studies, the proportion of FGFR3-mutated samples decreased with increasing stage (P<0.0001) and grade (P<0.0001). Fifty of the 56 CDKN2A homozygous deletions also included the neighboring centromeric gene CDKN2B and only 39 encompassed the neighboring telomeric gene MTAP. As both hemizygous and homozygous CDKN2A deletions always affected both P16^{INK4a} and P14^{ARF}, in this text, hemizygous and homozygous CDKN2A deletions therefore refer to the status of CDKN2A as a whole. They were no significant association between CDKN2A homozygous deletion and stage (P=0.168 or grade (P=0.311) when not taking into account the FGFR3 mutation status of the samples (FIG. 4).

High Frequency of CDKN2A Homozygous Deletions in FGFR3-Mutated Tumors and Association with Stage and Grade

Firstly, the frequency of CDKN2A homozygous deletion in FGFR3-mutated tumors and in wild-type FGFR3 tumors were compared. The percentage of CDKN2A homozygous deletions in FGFR3-mutated tumors (28%, 35/124) was significantly higher than those in FGFR3-wild-type tumors (13%, 21/164) (P=0.0015) (two-tailed Fisher's exact test).

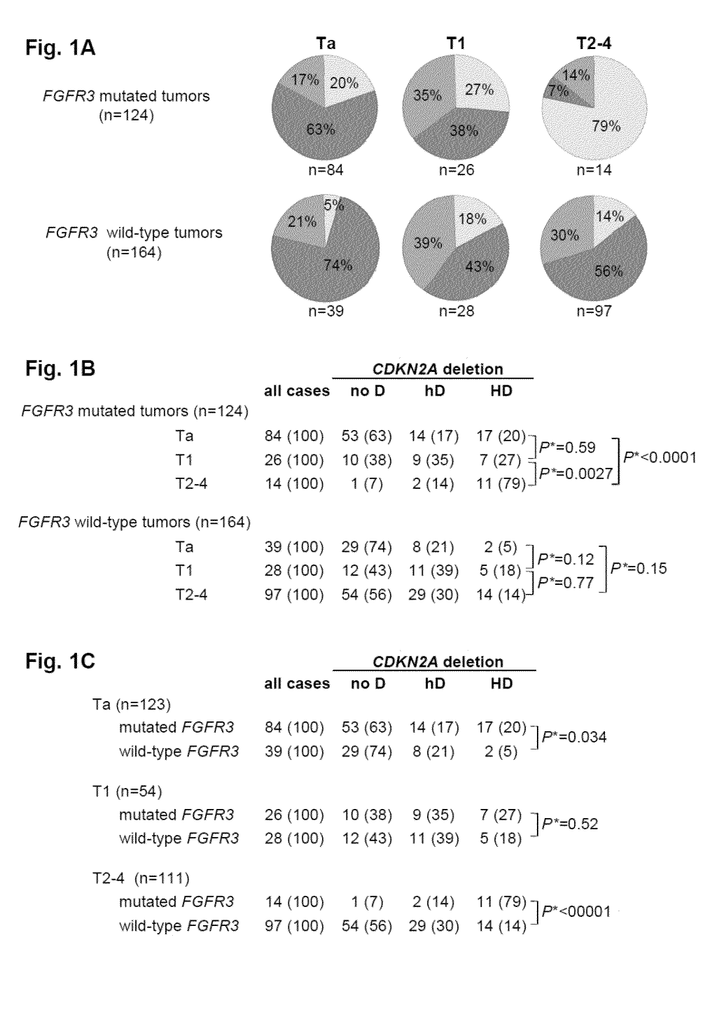

The association of CDKN2A homozygous deletion with stage in the FGFR3-mutated tumors and in the FGFR3-wild-type tumors was then assessed (FIG. 1). The inventors observed an important increase of CDKN2A homozygous deletion frequency as a function of stage in FGFR3-mutated tumors (20%, 17/84 in Ta; 38%, 7/26 in T1; and 79%, 11/14 in muscle-invasive tumors (T2-4)) (FIG. 1A). When performing a chi-squared test for trend (see Material and Methods), this increase was found to be highly significant (P<0.0001) whereas no significant association of CDKN2A homozygous deletion with stage (P=0.21) was found for FGFR3-wild-type tumors. Homozygous deletions were then compared between the different stages (Ta/T1, T1/T2-4 and Ta/T2-4) (FIG. 1B). For the FGFR3-mutated tumors, there was no significant difference between Ta and T1 tumors (P=0.59) but a highly significant difference between Ta and T2-4 tumors and between T1 and T2-4 tumors (P<0.0001 and P=0.0027 respectively) (two-tailed Fisher's exact test). In contrast, no significant increase of homozygous deletion was observed between the different stages in FGFR3-wild-type tumors (FIG. 1B). FIG. 1C compared CDKN2A homozygous deletions between FGFR3-mutated and FGFR3-wild-type tumors for each stage. The difference was slightly significant in Ta (P=0.034) but not in T1 tumors (P=0.52) whereas it was highly significant in muscle-invasive tumors (P<0.0001).

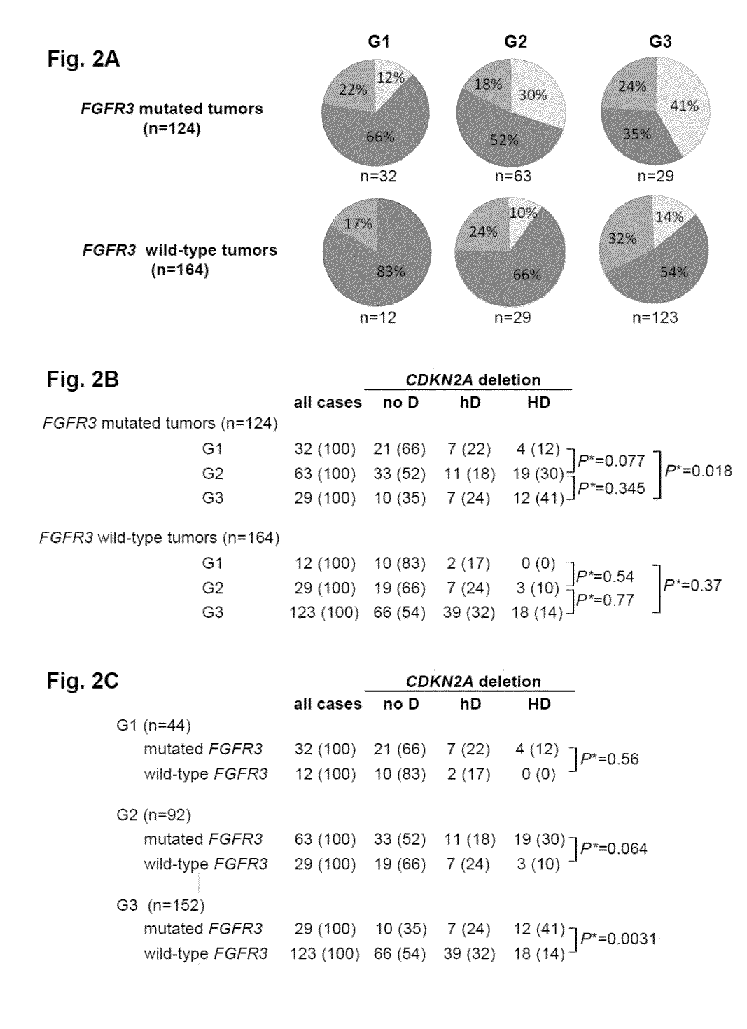

The inventors then assessed the association of CDKN2A homozygous deletions with grade in the FGFR3-mutated tumors and in the FGFR3-wild-type tumors. An increase of CDKN2A homozygous deletions as a function of grade (12%, 4/32 in G1; 30%, 19/63 in G2; and 41%, 12/29 in G3 tumors) was observed (FIG. 2A). When performing a chi-squared test for trend (see Material and Methods), this increase was found to be significant (P=0.012) whereas no significant association was found for FGFR3-wild-type tumors between CDKN2A homozygous deletion and grade (P=0.14). Homozygous deletions were then compared between the different grades (G1/G2, G2/G3 and G1/G3) (FIG. 2B). For the FGFR3-mutated tumors, the only significant difference was between G1 and G3 tumors (P=0.018) (two-tailed Fisher's exact test). No significant increase of homozygous deletions was observed between the different grades in FGFR3-wild-type tumors (FIG. 2B). FIG. 2C compared CDKN2A homozygous deletions between FGFR3-mutated and FGFR3-wild-type tumors for each grade. The difference was highly significant only in G3 tumors (P=0.0031).

CDKN2A Hemizygous and Homozygous Deletions are Associated with Progression of Non-Muscle-Invasive Bladder Tumors with FGFR3 Mutation

The high frequency of homozygous deletion of CDKN2A in muscle-invasive FGFR3-mutated bladder tumors led the inventors to investigate whether CDKN2A deletions affected the prognosis of patients with non-muscle-invasive tumors mutated in FGFR3. They did not only consider tumors which have lost two copies of the gene but also tumors which have lost one copy of CDKN2A as hemizygous tumors have already acquired one of the two hits for the full loss of the CDKN2A gene.

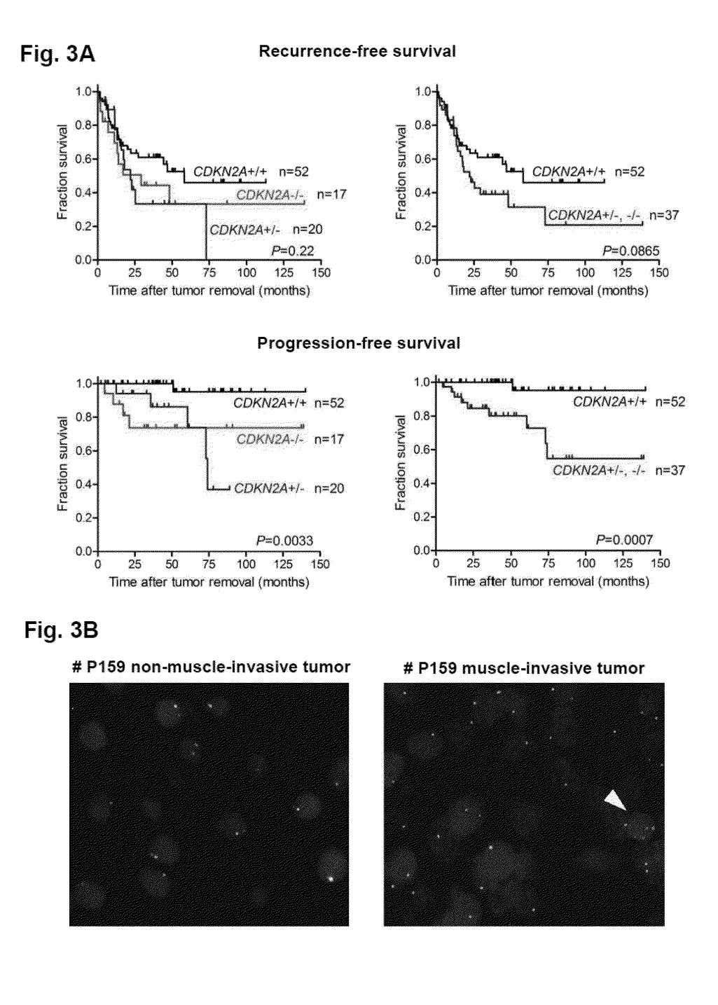

Kaplan-Meier analyses of recurrence and muscle-invasive progression were carried out in 89 patients with non-muscle-invasive FGFR3-mutated tumors for whom the follow-up was available (maximum follow up=11.7 years, median=3.5) (FIG. 3A). The data corresponding to these analyses are given in Table 2. Tumors which presented no deletion of the CDKN2A locus were compared by the log rank test with tumors lacking one or both copies of this locus (FIG. 3A). These 3 groups of tumors behaved similarly in terms of recurrence (P=0.22) (FIG. 3A left upper panel), but CDKN2A loss was significantly associated with muscle-invasive progression (P=0.0033) (FIG. 3A left lower panel). When considering two groups of tumors (tumors with no CDKN2A deletion and tumors with CDKN2A hemizygous or homozygous deletions), there was a trend for CDKN2A deletion to be associated with recurrence (P=0.0865) (FIG. 3A right upper panel) but CDKN2A deletion was highly associated with progression (P=0.0007) (FIG. 3A right lower panel). CDKN2A deletion was also highly associated with progression (P=0.0029, when comparing the three groups of tumors, P=0.0015 when considering two groups of tumors) when we considered among the 89 patients only the 76 patients for which the tumor that we analysed was the first tumor diagnosed.

Among the 10 patients who progressed, four presented in their non-muscle invasive tumor a homozygous CDKN2A deletion, five presented a hemizygous deletion and one showed a normal copy number of CDKN2A. No corresponding invasive progression sample was available for the case without deletion. For four of the five cases displaying a hemizygous deletion, the corresponding invasive progression sample being available, the inventors could determine whether the second copy of CDKN2A had been lost during progression. The CDKN2A copy number in these invasive progression samples was determined by MLPA (for cases P194, P223), or fluorescence in situ hybridization (for cases P159 and P288) when no DNA was available. Secondary loss of the remaining CDKN2A allele was observed in the muscle-invasive sample for two of the four cases, case P159 (FIG. 3B) and case P223. The propensity to progress of FGFR3mutated non-muscle-invasive tumors displaying CDKN2A deletion (homozygous or hemizygous) and the loss of the second allele of CDKN2A during the progression of hemizygous tumor is consistent with the high proportion of CDKN2A homozygous deletion observed in muscle-invasive FGFR3-mutated tumors.

### CONCLUSION

The inventors herein showed that CDKN2A homozygous deletions are significantly more frequent in FGFR3-mutated tumors compared to FGFR3-wild-type tumors. Moreover, they demonstrated that within the FGFR3-mutated tumor group, homozygous deletions are associated with muscle-invasive stages (T2-4). Consistent with this, progression-free survival analyses of non-muscle-invasive FGFR3-mutated bladder cancer patients show that CDKN2A losses are associated with progression to muscle invasion and/or metastasis.

Previous studies have pointed out CDKN2A homozygous deletions as the main mechanism of inactivation of the two oncoproteins (P16 and P14) coded by this gene. However no clear association between these deletions and stage or grade could be observed. In the present study if the inventors did not take into account the FGFR3 mutation status, they also could not observe any association between CDKN2A homozygous deletion and stage (CDKN2A homozygous deletions were 15.4%, 22.2%, 22.5% for Ta, T1, T2-4 tumors respectively). In contrast the association of CDKN2A homozygous deletions with stage was obvious within the FGFR3-mutated tumors (20%, 38%, 79% for Ta, T1, T2-4 tumors respectively; P<0.0001). The absence of association between CDKN2A deletion and the stage in all tumors (FGFR3-mutated and non-mutated tumors) can be explained by the high frequency of FGFR3 mutation (70%-75%) in Ta tumors and their low frequency in T2-T4 tumors (10-15%). Indeed as Ta tumors are mainly composed of FGFR3-mutated tumors, the frequency of CDKN2A homozygous deletion (15.4%) observed in the whole Ta tumors (FGFR3-mutated and non-mutated tumors) is close to those observed (20%) in the FGFR3-mutated Ta tumor subgroup. Conversely, as T2-4 tumors are mainly composed of wild-type FGFR3 tumors (85-90%), the percentage of CDKN2A homozygous deletion (22.5%) observed in the whole T2-4 tumors (FGFR3-mutated and not-mutated tumors) is close to those observed in the wild-type FGFR3 T2-4 tumor subgroup (14%).

FGFR3 mutations are considered as a marker of the Ta pathway. Present in 25% of hyperplastic lesions, FGFR3 mutations are the main alteration found so far in Ta tumors (70-75% of cases). They are usually maintained during recurrence and progression. The low frequency of FGFR3 mutation in muscle invasive tumors is in agreement with the low rate of Ta progression. The fact that homozygous CDKN2A deletion are associated with the muscle-invasive stages in FGFR3-mutated tumors, that Ta and T1 FGFR3-mutated tumors which harbor CDKN2A hemi- or homozygous losses tend to progress in favor of CDKN2A homozygous deletion as a key event in tumor progression in the Ta pathway. Consistent with the involvement of CDKN2A losses in tumor progression, the inventors observed for two FGFR3-mutated T1 tumors harboring hemizygous loss of CDKN2A, the loss of the remaining copy when they progressed to muscle invasive tumors. It is interesting to note, that the time to progression (51.2 months+/−26.6) of the 5 tumors displaying hemizygous deletion was longer than those (13.4 months+/−7.4) of the 4 tumors displaying homozygous deletion (P=0.07; Mann-Whitney nonparametric test).

TABLE 1
Anatomical and clinical parameters, FGFR3 mutation status
and CDKN2A copy number of the 288 samples studied

| | | | | FGFR3 | CDKN2A copy |
|---|---|---|---|---|---|
| Sample | Stage | Grade | Sample type | mutation | number |
| P1 | T4a | G3 | progression | wild-type | Hemizygous deletion |
| P2 | Ta | G1 | primary tumor | S249C | Normal |
| P3 | Ta | G3 | primary tumor | wild-type | Normal |
| P4 | T2 | G2 | primary tumor | wild-type | Normal |
| P5 | T4a | G3 | primary tumor | wild-type | Hemizygous deletion |
| P6 | T2 | G2 | primary tumor | S249C | Homozygous deletion |
| P7 | T3a | G3 | progression | wild-type | Hemizygous deletion |
| P8 | T1a | G3 | ND | wild-type | Hemizygous deletion |
| P9 | T4a | G3 | primary tumor | wild-type | Normal |
| P10 | T4a | G3 | primary tumor | wild-type | Normal |
| P11 | T4a | G3 | primary tumor | wild-type | Normal |
| P12 | T3 | G2 | ND | wild-type | Homozygous deletion |
| P13 | T3b | G2 | progression | S249C | Homozygous deletion |
| P14 | T4a | G2 | primary tumor | wild-type | Normal |
| P15 | T1b | G2 | recurrence | S249C | Homozygous deletion |
| P16 | Ta | G2 | primary tumor | S249C | Normal |
| P17 | T3b | G3 | primary tumor | wild-type | Normal |
| P18 | Ta | G3 | ND | wild-type | Hemizygous deletion |
| P19 | T4a | G3 | primary tumor | wild-type | Hemizygous deletion |
| P20 | T4a | G3 | recurrence | wild-type | Homozygous deletion |
| P21 | T4a | G3 | recurrence | wild-type | Normal |
| P22 | T4a | G3 | recurrence | wild-type | Normal |
| P23 | T3b | G3 | primary tumor | wild-type | Homozygous deletion |
| P24 | T3a | G3 | primary tumor | S249C | Homozygous deletion |
| P25 | T1b | G3 | primary tumor | wild-type | Homozygous deletion |
| P26 | T2 | G3 | primary tumor | wild-type | Normal |
| P27 | T1a | G2 | primary tumor | S249C, A391E | Normal |
| P28 | T1a | G1 | primary tumor | S373C | Normal |
| P29 | T4b | G3 | progression | wild-type | Homozygous deletion |
| P30 | T4a | G3 | primary tumor | wild-type | Normal |
| P31 | T4b | G3 | progression | wild-type | Hemizygous deletion |
| P32 | T4a | G3 | primary tumor | wild-type | Homozygous deletion |
| P33 | T4a | G3 | primary tumor | wild-type | Hemizygous deletion |
| P34 | T3b | G3 | primary tumor | Y375C | Homozygous deletion |
| P35 | T3a | G3 | primary tumor | wild-type | Normal |
| P36 | T3b | G3 | progression | wild-type | Hemizygous deletion |
| P37 | T3b | G3 | primary tumor | wild-type | Hemizygous deletion |
| P38 | T3b | G3 | primary tumor | wild-type | Normal |
| P39 | T3a | G3 | primary tumor | wild-type | Normal |
| P40 | T3a | G3 | primary tumor | wild-type | Normal |
| P41 | T3b | G3 | primary tumor | wild-type | Normal |
| P42 | T3a | G3 | primary tumor | wild-type | Normal |
| P43 | T3b | G3 | progression | wild-type | Normal |
| P44 | T4a | G3 | progression | wild-type | Homozygous deletion |
| P45 | T3b | G3 | progression | wild-type | Hemizygous deletion |
| P46 | T2 | G3 | primary tumor | wild-type | Normal |
| P47 | T2 | G3 | primary tumor | wild-type | Normal |
| P48 | T2 | G3 | progression | wild-type | Normal |
| P49 | T4b | G3 | primary tumor | wild-type | Normal |
| P50 | T2 | G3 | primary tumor | wild-type | Normal |
| P51 | T2 | G3 | primary tumor | S249C | Homozygous deletion |
| P52 | T2 | G3 | primary tumor | wild-type | Gain |
| P53 | T4a | G3 | primary tumor | wild-type | Hemizygous deletion |
| P54 | T1b | G3 | recurrence | wild-type | Hemizygous deletion |
| P55 | T1b | G3 | recurrence | S249C | Normal |
| P56 | T2 | G3 | ND | wild-type | Normal |
| P57 | T1a | G3 | primary tumor | R248C | Hemizygous deletion |
| P58 | Ta | G2 | primary tumor | S249C | Hemizygous deletion |
| P59 | T1a | G2 | primary tumor | R248C | Homozygous deletion |
| P60 | Ta | G3 | primary tumor | wild-type | Normal |
| P61 | T1 | G3 | primary tumor | S249C | Homozygous deletion |
| P62 | T1a | G2 | primary tumor | Y375C | Homozygous deletion |
| P63 | T1b | G3 | primary tumor | wild-type | Hemizygous deletion |
| P64 | Ta | G2 | primary tumor | Y375C | Homozygous deletion |
| P65 | T1a | G3 | primary tumor | G372C | Normal |
| P66 | T2 | G3 | primary tumor | wild-type | Homozygous deletion |
| P67 | T3a | G3 | progression | S249C | Homozygous deletion |
| P68 | T4b | G2 | progression | S249C | Homozygous deletion |
| P69 | T3b | G3 | primary tumor | wild-type | Hemizygous deletion |
| P70 | T3a | G3 | primary tumor | wild-type | Hemizygous deletion |
| P71 | T3a | G3 | primary tumor | wild-type | Homozygous deletion |
| P72 | T3b | G3 | primary tumor | wild-type | Homozygous deletion |
| P73 | T4a | G3 | primary tumor | wild-type | Homozygous deletion |
| P74 | T3a | G3 | primary tumor | wild-type | Normal |
| P75 | T2 | G3 | primary tumor | wild-type | Normal |
| P76 | T3b | G3 | progression | wild-type | Hemizygous deletion |
| P77 | T3a | G3 | primary tumor | wild-type | Homozygous deletion |
| P78 | T3a | G3 | primary tumor | wild-type | Homozygous deletion |
| P79 | T3b | G3 | primary tumor | wild-type | Normal |
| P80 | T4b | G3 | primary tumor | wild-type | Gain |
| P81 | Ta | G2 | primary tumor | wild-type | Homozygous deletion |
| P82 | Ta | G2 | primary tumor | R248C | Normal |
| P83 | T1a | G3 | primary tumor | wild-type | Hemizygous deletion |
| P84 | T2 | G3 | primary tumor | wild-type | Normal |
| P85 | T1 | G2 | primary tumor | S249C | Homozygous deletion |
| P86 | T3a | G3 | primary tumor | wild-type | Normal |
| P87 | Ta | G2 | primary tumor | Y375C | Homozygous deletion |
| P88 | T3a | G3 | primary tumor | wild-type | Hemizygous deletion |
| P89 | T3b | G3 | primary tumor | S249C | Normal |
| P90 | T1a | G3 | primary tumor | wild-type | Normal |
| P91 | T4 | G3 | progression | R248C | Homozygous deletion |
| P92 | T3b | G3 | primary tumor | wild-type | Hemizygous deletion |
| P93 | T1b | G3 | primary tumor | wild-type | Normal |
| P94 | T2 | G3 | progression | wild-type | Hemizygous deletion |
| P95 | T1a | G3 | primary tumor | wild-type | Hemizygous deletion |
| P96 | T4 | G3 | primary tumor | wild-type | Normal |
| P97 | T1a | G3 | primary tumor | wild-type | Hemizygous deletion |
| P98 | T3b | G3 | primary tumor | wild-type | Normal |
| P99 | T1a | G3 | primary tumor | wild-type | Hemizygous deletion |
| P100 | T2 | G3 | primary tumor | wild-type | Normal |
| P101 | T2 | G3 | primary tumor | wild-type | Normal |
| P102 | T1 | G3 | primary tumor | wild-type | Homozygous deletion |
| P103 | T1a | G3 | primary tumor | wild-type | Hemizygous deletion |
| P104 | T2 | G3 | primary tumor | wild-type | Normal |
| P105 | T1a | G3 | primary tumor | S249C, R248C | Normal |
| P106 | T1 | G3 | primary tumor | wild-type | Normal |
| P107 | T3a | G3 | primary tumor | wild-type | Hemizygous deletion |
| P108 | T2 | G3 | primary tumor | wild-type | Normal |
| P109 | T2 | G3 | primary tumor | wild-type | Hemizygous deletion |
| P110 | T2 | G3 | primary tumor | wild-type | Normal |
| P111 | Ta | G2 | primary tumor | S249C | Normal |
| P112 | Ta | G1 | primary tumor | wild-type | Normal |
| P113 | Ta | G3 | primary tumor | Y375C | Normal |
| P114 | T1a | G2 | ND | Y375C | Hemizygous deletion |
| P115 | T1a | G3 | primary tumor | S249C | Normal |
| P116 | T1b | G3 | primary tumor | wild-type | Hemizygous deletion |
| P117 | T1b | G3 | primary tumor | wild-type | Normal |
| P118 | T1a | G3 | ND | S249C | Hemizygous deletion |
| P119 | Ta | G3 | primary tumor | wild-type | Normal |
| P120 | T2 | G2 | primary tumor | wild-type | Hemizygous deletion |
| P121 | T1a | G3 | recurrence | wild-type | Homozygous deletion |
| P122 | T2 | G3 | primary tumor | wild-type | Normal |
| P123 | T4a | G3 | primary tumor | wild-type | Hemizygous deletion |
| P124 | Ta | G2 | primary tumor | Y375C | Normal |
| P125 | T4a | G3 | primary tumor | wild-type | Normal |
| P126 | T4a | G3 | primary tumor | wild-type | Homozygous deletion |
| P127 | T2 | G3 | primary tumor | wild-type | Gain |
| P128 | T2 | G3 | primary tumor | wild-type | Hemizygous deletion |
| P129 | T2 | G3 | primary tumor | wild-type | Normal |
| P130 | T4 | G3 | primary tumor | wild-type | Normal |
| P131 | T3a | G3 | primary tumor | wild-type | Normal |
| P132 | T4 | G3 | primary tumor | wild-type | Homozygous deletion |
| P133 | T3 | G3 | primary tumor | wild-type | Normal |
| P134 | T2 | G3 | primary tumor | wild-type | Normal |
| P135 | Ta | G2 | recurrence | wild-type | Hemizygous deletion |
| P136 | Ta | G1 | primary tumor | wild-type | Normal |
| P137 | T1b | G2 | primary tumor | S249C | Normal |
| P138 | T1a | G3 | primary tumor | Y375C | Hemizygous deletion |
| P139 | Ta | G2 | recurrence | S249C | Normal |
| P140 | Ta | G2 | primary tumor | Y375C | Hemizygous deletion |
| P141 | Ta | G2 | primary tumor | K652E | Normal |
| P142 | Ta | G2 | primary tumor | S249C | Homozygous deletion |
| P143 | Ta | G1 | primary tumor | wild-type | Normal |
| P144 | Ta | G2 | primary tumor | S249C | Normal |
| P145 | Ta | G1 | primary tumor | S249C | Normal |
| P146 | Ta | G2 | primary tumor | Y375C | Normal |
| P147 | Ta | G2 | primary tumor | R248C | Normal |
| P148 | Ta | G2 | primary tumor | S249C | Normal |
| P149 | T4a | G3 | primary tumor | wild-type | Normal |
| P150 | Ta | G2 | ND | S249C, R248C | Normal |
| P151 | T1a | G3 | primary tumor | wild-type | Normal |
| P152 | Ta | G2 | ND | wild-type | Normal |
| P153 | Ta | G2 | primary tumor | S249C, R248C | Normal |
| P154 | Ta | G3 | primary tumor | wild-type | Gain |
| P155 | Ta | G2 | primary tumor | S249C | Hemizygous deletion |
| P156 | T2 | G3 | primary tumor | wild-type | Normal |
| P157 | T3a | G2 | primary tumor | S249C | Hemizygous deletion |
| P158 | T3a | G3 | primary tumor | wild-type | Normal |
| P159 | Ta | G2 | recurrence | S249C | Hemizygous deletion |
| P160 | T2 | G3 | primary tumor | S249C | Homozygous deletion |
| P161 | Ta | G3 | primary tumor | S249C | Homozygous deletion |
| P162 | T2 | G3 | primary tumor | wild-type | Hemizygous deletion |
| P163 | T3b | G3 | primary tumor | wild-type | Normal |
| P164 | T3a | G3 | primary tumor | wild-type | Normal |
| P165 | T3a | G3 | primary tumor | wild-type | Hemizygous deletion |
| P166 | Ta | G2 | recurrence | G372C | Homozygous deletion |
| P167 | T1a | G3 | recurrence | S249C | Homozygous deletion |
| P168 | T3b | G2 | progression | wild-type | Hemizygous deletion |
| P169 | T3a | G3 | primary tumor | wild-type | Normal |
| P170 | T2 | G2 | progression | wild-type | Hemizygous deletion |
| P171 | T3 | G2 | primary tumor | wild-type | Normal |
| P172 | Ta | G1 | primary tumor | S249C | Hemizygous deletion |
| P173 | T3a | G3 | primary tumor | wild-type | Normal |
| P174 | Ta | G2 | primary tumor | S249C | Normal |
| P175 | T1a | G3 | primary tumor | wild-type | Hemizygous deletion |
| P176 | T1a | G2 | primary tumor | wild-type | Normal |
| P177 | T1a | G3 | primary tumor | wild-type | Normal |
| P178 | T1a | G3 | primary tumor | S249C | Normal |
| P179 | T2 | G3 | primary tumor | wild-type | Normal |
| P180 | T2 | G3 | primary tumor | wild-type | Hemizygous deletion |
| P181 | T1a | G3 | primary tumor | wild-type | Normal |
| P182 | T1a | G3 | primary tumor | wild-type | Homozygous deletion |
| P183 | T1a | G3 | primary tumor | wild-type | Homozygous deletion |
| P184 | Ta | G2 | primary tumor | S249C | Normal |
| P185 | T1b | G3 | primary tumor | wild-type | Hemizygous deletion |
| P186 | Ta | G3 | recurrence | wild-type | Hemizygous deletion |
| P187 | T1 | G3 | primary tumor | wild-type | Normal |
| P188 | T1a | G3 | primary tumor | wild-type | Normal |
| P189 | T2 | G3 | primary tumor | wild-type | Hemizygous deletion |
| P190 | T1a | G3 | recurrence | R248C | Normal |
| P191 | T1a | G2 | recurrence | S249C | Hemizygous deletion |
| P192 | T1b | G3 | primary tumor | wild-type | Normal |
| P193 | Ta | G2 | recurrence | R248C | Homozygous deletion |
| P194 | T1a | G3 | primary tumor | Y375C | Hemizygous deletion |
| P195 | Ta | G3 | primary tumor | R248C | Normal |
| P196 | Ta | G2 | primary tumor | S249C | Normal |
| P197 | Ta | G1 | primary tumor | G372C | Normal |
| P198 | Ta | G1 | primary tumor | S249C | Normal |
| P199 | Ta | G2 | primary tumor | S249C | Homozygous deletion |
| P200 | T1a | G3 | primary tumor | R248C | Normal |
| P201 | T1a | G3 | primary tumor | S249C | Homozygous deletion |
| P202 | T1b | G3 | primary tumor | wild-type | Normal |
| P203 | Ta | G1 | ND | S249C, R248C | Normal |
| P204 | T2 | G3 | primary tumor | wild-type | Hemizygous deletion |
| P205 | Ta | G2 | primary tumor | S249C, R248C | Normal |
| P206 | Ta | G2 | primary tumor | S249C | Normal |
| P207 | Ta | G1 | primary tumor | wild-type | Normal |
| P208 | Ta | G2 | primary tumor | S249C | Homozygous deletion |
| P209 | Ta | G2 | primary tumor | wild-type | Normal |
| P210 | T1a | G3 | ND | Y375C | Hemizygous deletion |
| P211 | T3a | G3 | primary tumor | wild-type | Hemizygous deletion |
| P212 | Ta | G2 | primary tumor | wild-type | Normal |
| P213 | Ta | G2 | primary tumor | S249C | Normal |
| P214 | Ta | G2 | primary tumor | S249C | Normal |
| P215 | Ta | G2 | primary tumor | S249C | Hemizygous deletion |
| P216 | Ta | G1 | primary tumor | S249C, K652E | Normal |
| P217 | Ta | G2 | recurrence | S249C | Hemizygous deletion |
| P218 | Ta | G1 | recurrence | wild-type | Normal |
| P219 | Ta | G1 | primary tumor | S249C | Normal |
| P220 | Ta | G2 | recurrence | S249C | Hemizygous deletion |
| P221 | T2 | G3 | recurrence | Y375C | Hemizygous deletion |
| P222 | T3b | G3 | progression | S249C | Homozygous deletion |
| P223 | T1a | G3 | recurrence | S249C | Hemizygous deletion |
| P224 | T4a | G3 | progression | S249C | Homozygous deletion |
| P225 | Ta | G1 | primary tumor | Y375C | Normal |
| P226 | Ta | G1 | primary tumor | S249C | Normal |
| P227 | Ta | G1 | primary tumor | S249C | Normal |
| P228 | Ta | G1 | primary tumor | S249C | Hemizygous deletion |
| P229 | Ta | G1 | primary tumor | wild-type | Gain |
| P230 | Ta | G2 | primary tumor | wild-type | Hemizygous deletion |
| P231 | Ta | G1 | primary tumor | Y375C | Hemizygous deletion |
| P232 | Ta | G2 | primary tumor | Y375C | Normal |
| P233 | Ta | G1 | primary tumor | S249C | Hemizygous deletion |
| P234 | Ta | G2 | primary tumor | wild-type | Normal |
| P235 | Ta | G2 | primary tumor | wild-type | Normal |
| P236 | Ta | G1 | primary tumor | S249C | Homozygous deletion |
| P237 | Ta | G2 | primary tumor | wild-type | Gain |
| P238 | Ta | G2 | primary tumor | S249C | Normal |
| P239 | Ta | G2 | primary tumor | wild-type | Normal |
| P240 | Ta | G2 | primary tumor | wild-type | Gain |
| P241 | Ta | G1 | primary tumor | S249C | Hemizygous deletion |
| P242 | Ta | G1 | primary tumor | S249C | Gain |
| P243 | Ta | G1 | primary tumor | wild-type | Hemizygous deletion |
| P244 | Ta | G2 | primary tumor | wild-type | Hemizygous deletion |
| P245 | Ta | G1 | primary tumor | S249C | Hemizygous deletion |
| P246 | Ta | G1 | primary tumor | S249C | Hemizygous deletion |
| P247 | Ta | G1 | primary tumor | S249C | Gain |
| P248 | Ta | G1 | primary tumor | S249C | Normal |
| P249 | Ta | G1 | primary tumor | R248C | Normal |
| P250 | Ta | G2 | primary tumor | wild-type | Gain |
| P251 | Ta | G1 | primary tumor | wild-type | Hemizygous deletion |
| P252 | Ta | G1 | primary tumor | S249C | Gain |
| P253 | Ta | G1 | primary tumor | Y375C | Normal |
| P254 | Ta | G2 | primary tumor | R248C | Homozygous deletion |
| P255 | Ta | G2 | primary tumor | wild-type | Homozygous deletion |
| P256 | Ta | G2 | primary tumor | wild-type | Normal |
| P257 | Ta | G1 | primary tumor | K652E | Normal |
| P258 | Ta | G1 | primary tumor | S249C | Gain |
| P259 | Ta | G2 | primary tumor | S249C | Homozygous deletion |
| P260 | Ta | G2 | primary tumor | S249C | Normal |
| P261 | Ta | G1 | primary tumor | R248C | Homozygous deletion |
| P262 | Ta | G1 | primary tumor | wild-type | Normal |
| P263 | Ta | G2 | primary tumor | wild-type | Normal |
| P264 | Ta | G2 | primary tumor | S249C | Normal |
| P265 | Ta | G1 | primary tumor | R248C | Normal |
| P266 | Ta | G1 | primary tumor | Y375C | Normal |
| P267 | Ta | G2 | primary tumor | S373C | Homozygous deletion |
| P268 | Ta | G2 | primary tumor | wild-type | Normal |
| P269 | Ta | G1 | primary tumor | wild-type | Normal |
| P270 | Ta | G2 | primary tumor | S249C | Homozygous deletion |
| P271 | Ta | G2 | primary tumor | wild-type | Normal |
| P272 | Ta | G2 | primary tumor | R248C | Homozygous deletion |
| P273 | Ta | G2 | primary tumor | S249C | Normal |
| P274 | Ta | G1 | primary tumor | S249C | Homozygous deletion |
| P275 | Ta | G2 | primary tumor | wild-type | Normal |
| P276 | Ta | G2 | primary tumor | wild-type | Normal |
| P277 | Ta | G2 | primary tumor | Y375C | Normal |
| P278 | Ta | G2 | primary tumor | R248C | Normal |
| P279 | Ta | G2 | primary tumor | wild-type | Hemizygous deletion |
| P280 | Ta | G1 | primary tumor | wild-type | Normal |
| P281 | Ta | G1 | primary tumor | wild-type | Normal |
| P282 | Ta | G2 | primary tumor | K652E | Normal |
| P283 | Ta | G2 | primary tumor | S249C | Normal |
| P284 | Ta | G2 | primary tumor | S249C | Normal |
| P285 | Ta | G2 | primary tumor | S249C | Normal |
| P286 | Ta | G2 | primary tumor | S249C | Normal |
| P287 | Ta | G1 | primary tumor | S249C | Homozygous deletion |
| P288 | T1 | G2 | ND | R248C | Hemizygous deletion |

TABLE 2
Follow-up data used for Kaplan-Meier analysis. Durations are indicated in months.

| | | | | | | Follow-up | |
|---|---|---|---|---|---|---|---|
| | | | | FGFR3 | | time | |
| Sample | Stage | Grade | Primary tumors | mutation | CDKN2A copy number | (months) | Last known status |
| P59 | T1a | G2 | primary tumor | R248C | Homozygous deletion | 5.0 | Alive tumor-free |
| P201 | T1a | G3 | primary tumor | S249C | Homozygous deletion | 31.4 | Alive tumor-free |
| P161 | Ta | G3 | primary tumor | S249C | Homozygous deletion | 33.0 | Alive tumor-free |
| P287 | Ta | G1 | primary tumor | S249C | Homozygous deletion | 39.3 | Alive |
| P87 | Ta | G2 | primary tumor | Y375C | Homozygous deletion | 52 | Alive tumor-free |
| P61 | T1 | G3 | primary tumor | S249C | Homozygous deletion | 87.0 | Alive tumor-free |
| P166 | Ta | G2 | recurrence | G372C | Homozygous deletion | 138.9 | Death from other cause |
| P193 | Ta | G2 | recurrence | R248C | Homozygous deletion | 14.6 | Alive tumor-free |
| P236 | Ta | G1 | primary tumor | S249C | Homozygous deletion | 18.3 | Alive |
| P142 | Ta | G2 | primary tumor | S249C | Homozygous deletion | 34.0 | Alive tumor-free |
| P254 | Ta | G2 | primary tumor | R248C | Homozygous deletion | 53.5 | Alive |
| P15 | T1b | G2 | recurrence | S249C | Homozygous deletion | 91.0 | Alive tumor-free |
| P199 | Ta | G2 | primary tumor | S249C | Homozygous deletion | 137.5 | Alive tumor-free |
| P261 | Ta | G1 | primary tumor | R248C | Homozygous deletion | 11.9 | Alive |
| P64 | Ta | G2 | primary tumor | Y375C | Homozygous deletion | 16 | Death from cancer |
| P62 | T1a | G2 | primary tumor | Y375C | Homozygous deletion | 34 | Death from cancer |
| P85 | T1 | G2 | primary tumor | S249C | Homozygous deletion | 53.0 | Death from cancer |
| P220 | Ta | G2 | recurrence | S249C | Hemizygous deletion | 5 | Alive tumor-free |
| P155 | Ta | G2 | primary tumor | S249C | Hemizygous deletion | 5 | Alive tumor-free |
| P191 | T1a | G2 | recurrence | S249C | Hemizygous deletion | 11 | Death from other cause |
| P140 | Ta | G2 | primary tumor | Y375C | Hemizygous deletion | 23 | Alive tumor-free |
| P217 | Ta | G2 | recurrence | S249C | Hemizygous deletion | 24 | Alive tumor-free |
| P228 | Ta | G1 | primary tumor | S249C | Hemizygous deletion | 37.1 | Alive |
| P241 | Ta | G1 | primary tumor | S249C | Hemizygous deletion | 44.9 | Alive |
| P138 | T1a | G3 | primary tumor | Y375C | Hemizygous deletion | 48 | Alive tumor-free |
| P215 | Ta | G2 | primary tumor | S249C | Hemizygous deletion | 17 | Alive tumor-free |
| P172 | Ta | G1 | primary tumor | S249C | Hemizygous deletion | 33 | Alive tumor-free |
| P233 | Ta | G1 | primary tumor | S249C | Hemizygous deletion | 40.0 | Alive |
| P245 | Ta | G1 | primary tumor | S249C | Hemizygous deletion | 60.6 | Alive |
| P246 | Ta | G1 | primary tumor | S249C | Hemizygous deletion | 61.6 | Alive |
| P57 | T1a | G3 | primary tumor | R248C | Hemizygous deletion | 78 | Alive tumor-free |
| P58 | Ta | G2 | primary tumor | S249C | Hemizygous deletion | 89 | Alive tumor-free |
| P159* | Ta | G2 | primary tumor | S249C | Hemizygous deletion | 61 | Death from cancer |
| P231 | Ta | G1 | primary tumor | Y375C | Hemizygous deletion | 74.5 | Alive |
| P223* | T1a | G3 | recurrence | S249C | Hemizygous deletion | 80 | Death from cancer |
| P194* | T1a | G3 | primary tumor | Y375C | Hemizygous deletion | 21.4 | Alive tumor-free |
| P288* | T1 | G2 | NO | R248C | Hemizygous deletion | 44.9 | Alive with cancer |
| P253 | Ta | G1 | primary tumor | Y375C | Normal | 6.7 | Alive |
| P214 | Ta | G2 | primary tumor | S249C | Normal | 7 | Alive with cancer |
| P16 | Ta | G2 | primary tumor | S249C | Normal | 10 | Alive tumor-free |
| P219 | Ta | G1 | primary tumor | S249C | Normal | 16 | Alive tumor-free |
| P216 | Ta | G1 | primary tumor | S249C, K652E | Normal | 17 | Alive tumor-free |
| P260 | Ta | G2 | primary tumor | S249C | Normal | 20.3 | Alive |
| P174 | Ta | G2 | primary tumor | S249C | Normal | 26 | Aiive tumor-free |
| P184 | Ta | G2 | primary tumor | S249C | Normal | 26 | Alive tumor-free |
| P124 | Ta | G2 | primary tumor | Y375C | Normal | 26 | Death from other cause |
| P203 | Ta | G1 | ND | S249C, R248C | Normal | 34 | Alive tumor-free |
| P282 | Ta | G2 | primary tumor | K652E | Normal | 37.7 | Alive |
| P55 | T1b | G3 | recurrence | S249C | Normal | 39 | Alive tumor-free |
| P2 | Ta | G1 | primary tumor | S249C | Normal | 39 | Alive tumor-free |
| P153 | Ta | G2 | primary tumor | S249C, R248C | Normal | 40 | Alive tumor-free |
| P283 | Ta | G2 | primary tumor | S249C | Normal | 40.1 | Alive |
| P285 | Ta | G2 | primary tumor | S249C | Normal | 40.7 | Alive |
| P249 | Ta | G1 | primary tumor | R248C | Normal | 40.8 | Alive |
| P213 | Ta | G2 | primary tumor | S249C | Normal | 50 | Alive tumor-free |
| P113 | Ta | G3 | primary tumor | Y375C | Normal | 51 | Alive tumor-free |
| P257 | Ta | G1 | primary tumor | K652E | Normal | 52.0 | Alive |
| P144 | Ta | G2 | primary tumor | S249C | Normal | 58 | Alive tumor-free |
| P115 | T1a | G3 | primary tumor | S249C | Normal | 78 | Alive tumor-free |
| P82 | Ta | G2 | primary tumor | R248C | Normal | 83 | Alive tumor-free |
| P200 | T1a | G3 | primary tumor | R248C | Normal | 84 | Alive tumor-free |
| P195 | Ta | G3 | primary tumor | R248C | Normal | 85 | Alive tumor-free |
| P198 | Ta | G1 | primary tumor | S249C | Normal | 95 | Alive tumor-free |
| P148 | Ta | G2 | primary tumor | S249C | Normal | 96 | Alive tumor-free |
| P146 | Ta | G2 | primary tumor | Y375C | Normal | 113 | Alive tumor-free |
| P190 | T1a | G3 | recurrence | R248C | Normal | 2 | Alive tumor-free |
| P28 | T1a | G1 | primary tumor | S371C | Normal | 17 | Alive tumor-free |
| P178 | T1a | G3 | primary tumor | S249C | Normal | 31 | Alive tumor-free |
| P111 | Ta | G2 | primary tumor | S249C | Normal | 34 | Alive tumor-free |
| P27 | T1a | G2 | primary tumor | S249C, A391E | Normal | 37 | Alive tumor-free |
| P150 | Ta | G2 | ND | S249C, R248C | Normal | 37 | Alive tumor-free |
| P139 | Ta | G2 | recurrence | S249C | Normal | 39 | Alive tumor-free |
| P284 | Ta | G2 | primary tumor | S249C | Normal | 41.7 | Alive |
| P65 | T1a | G3 | primary tumor | G372C | Normal | 42 | Alive tumor-free |
| P205 | Ta | G2 | primary tumor | S249C, R248C | Normal | 53 | Alive tumor-free |
| P105 | T1a | G3 | primary tumor | S249C, R248C | Normal | 57 | Alive tumor-free |
| P238 | Ta | G2 | primary tumor | S249C | Normal | 58.6 | Alive |
| P145 | Ta | G1 | primary tumor | S249C | Normal | 75 | Alive tumor-free |
| P226 | Ta | G1 | primary tumor | S249C | Normal | 75.3 | Alive |
| P227 | Ta | G1 | primary tumor | S249C | Normal | 79.0 | Alive |
| P147 | Ta | G2 | primary tumor | R248C | Normal | 90 | Aiive tumor-free |
| P206 | Ta | G2 | primary tumor | S249C | Normal | 91 | Alive tumor-free |
| P197 | Ta | G1 | primary tumor | G372C | Normal | 103 | Aiive tumor-free |
| P196 | Ta | G2 | primary tumor | S249C | Normal | 140 | Alive tumor-free |
| P137 | Tib | G2 | primary tumor | S249C | Normal | 72 | Alive tumor-free |
| P242 | Ta | G1 | primary tumor | S249C | Gain | 61.7 | Alive |
| P247 | Ta | G1 | primary tumor | S249C | Gain | 44.3 | Alive |
| P258 | Ta | G1 | primary tumor | S249C | Gain | 41.9 | Alive |
| P252 | Ta | G1 | primary tumor | S249C | Gain | 46.4 | Alive |

| | | | Time to first | Time to first | Type of |
|---|---|---|---|---|---|
| Sample | Recurrence | Progression | recurrence | progression | progression |
| P59 | no | no | | | |
| P201 | no | no | | | |
| P161 | no | no | | | |
| P287 | no | no | | | |
| P87 | no | no | | | |
| P61 | no | no | | | |
| P166 | no | no | | | |
| P193 | yes | no | 3.3 | | |
| P236 | yes | no | 13.2 | | |
| P142 | yes | no | 29.2 | | |
| P254 | yes | no | 13.9 | | |
| P15 | yes | no | 1.9 | | |
| P199 | yes | no | 48.2 | | |
| P261 | yes | yes | 7.1 | 10.5 | Muscle-invasive tumor |
| P64 | yes | yes | 0.7 | 4.6 | Muscle-invasive tumor |
| P62 | yes | yes | 17.2 | 17.2 | Muscle-invasive tumor |
| P85 | yes | yes | 11.2 | 21.3 | Muscle-invasive tumor |
| P220 | no | no | | | |
| P155 | no | no | | | |
| P191 | no | no | | | |
| P140 | no | no | | | |
| P217 | no | no | | | |
| P228 | no | no | | | |
| P241 | no | no | | | |
| P138 | no | no | | | |
| P215 | yes | no | 11.5 | | |
| P172 | yes | no | 17.5 | | |
| P233 | yes | no | 23.0 | | |
| P245 | yes | no | 25.3 | | |
| P246 | yes | no | 18.1 | | |
| P57 | yes | no | 5.9 | | |
| P58 | yes | no | 13.0 | | |
| P159* | yes | yes | 22.1 | 60.6 | Metastasis |
| P231 | yes | yes | 15.8 | 74.1 | Metastasis |
| P223* | yes | yes | 73 | 73 | Muscle-invasive tumor |
| P194* | yes | yes | 1.7 | 12.5 | Muscle-invasive tumor |
| P288* | yes | yes | 11.6 | 35.7 | Muscle-invasive tumor |
| P253 | no | no | | | |
| P214 | no | no | | | |
| P16 | no | no | | | |
| P219 | no | no | | | |
| P216 | no | no | | | |
| P260 | no | no | | | |
| P174 | no | no | | | |
| P184 | no | no | | | |
| P124 | no | no | | | |
| P203 | no | no | | | |
| P282 | no | no | | | |
| P55 | no | no | | | |
| P2 | no | no | | | |
| P153 | no | no | | | |
| P283 | no | no | | | |
| P285 | no | no | | | |
| P249 | no | no | | | |
| P213 | no | no | | | |
| P113 | no | no | | | |
| P257 | no | no | | | |
| P144 | no | no | | | |
| P115 | no | no | | | |
| P82 | no | no | | | |
| P200 | no | no | | | |
| P195 | no | no | | | |
| P198 | no | no | | | |
| P148 | no | no | | | |
| P146 | no | no | | | |
| P190 | yes | no | 1.0 | | |
| P28 | yes | no | 1.3 | | |
| P178 | yes | no | 7.9 | | |
| P111 | yes | no | 7.2 | | |
| P27 | yes | no | 22.3 | | |
| P150 | yes | no | 8.8 | | |
| P139 | yes | no | 7.0 | | |
| P284 | yes | no | 15.1 | | |
| P65 | yes | no | 6.3 | | |
| P205 | yes | no | 13.4 | | |
| P105 | yes | no | 4.8 | | |
| P238 | yes | no | 7.2 | | |
| P145 | yes | no | 58.3 | | |
| P226 | yes | no | 9.5 | | |
| P227 | yes | no | 20.1 | | |
| P147 | yes | no | 3.2 | | |
| P206 | yes | no | 27.3 | | |
| P197 | yes | no | 14.9 | | |
| P196 | yes | no | 14.0 | | |
| P137 | yes | yes | 46.9 | 51.0 | Muscle-invasive tumor |
| P242 | yes | no | 13.6 | | |
| P247 | yes | no | 44.3 | | |
| P258 | no | no | | | |
| P252 | no | no | | | |

*Patients for which the corresponding invasive progression sample was analyzed for CDKN2A copy number.

### EXAMPLARY ASPECTS OF THE INVENTION

1. A method for predicting clinical outcome of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative of a non-muscle invasive bladder cancer with FGFR3 activating mutation with a poor prognosis.

2. The method according to aspect 1, wherein a poor prognosis is a shorter progression free survival and/or an increased metastasis occurrence and/or a decreased patient survival, preferably a shorter progression free survival.

3. A method for selecting a subject affected with a non-muscle invasive bladder cancer with an activating mutation in FGFR3 gene for an anti-tumoral therapy, or for determining whether a subject affected with a non-muscle invasive bladder cancer with an activating mutation in FGFR3 gene is susceptible to benefit from an anti-tumoral therapy, wherein the method comprises detecting a loss-of-function mutation in CDKN2A gene in bladder cancer cells from the subject, the presence of a loss-of-function mutation in the CDKN2A gene being indicative that an anti-tumoral therapy is required for the subject affected with the non-muscle invasive bladder cancer with an activating mutation in FGFR3 gene.

4. The method according to aspect 3, wherein the anti-tumoral therapy is an adjuvant or neoadjuvant therapy.

5. The method according to aspect 3, wherein the anti-tumoral therapy is immunotherapy, preferably BCG therapy

6. The method according to aspect 3, wherein the anti-tumoral therapy is partial or radical cystectomy.

7. A method for providing information for determining follow-up strategy of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative that an intensive follow-up is required for the subject affected with the non-muscle invasive bladder cancer with an activating mutation in FGFR3 gene.

8. The method according to any of aspects 1 to 7, wherein the loss-of-function mutation in CDKN2A gene is a hemizygous or homozygous deletion of the CDKN2A gene.

9. The method according to any of aspects 1 to 8, further comprising detecting a hemizygous or homozygous deletion at chromosome 11p region.

10. The method according to any of aspects 1 to 9, wherein the activating mutation in FGFR3 gene is selected from the group consisting of R248C, S249C, P250R, G372C, S373C, Y375C, G382R, A391E, K652E, K652Q, K652M and K652T, and a combination thereof.

11. The method according to any of aspects 1 to 10, further comprising assessing at least one other cancer or prognosis markers such as tumor stage, grade, number of tumors, prior recurrence rate, mitotic index, tumor size, HJURP expression level, HP1α expression level or expression of proliferation markers such as Ki67, MCM2, CAF-1 p60 and CAF-1 p150.

12. A kit (a) for predicting or monitoring clinical outcome of a subject affected with a non-muscle invasive bladder cancer; and/or (b) for selecting a subject affected with a non-muscle invasive bladder cancer for an anti-tumoral therapy, or for determining whether a subject affected with a non-muscle invasive bladder cancer is susceptible to benefit from an anti-tumoral therapy; and/or (c) for providing information for determining follow-up strategy of a subject affected with a non-muscle invasive bladder cancer, wherein the kit comprises (i) at least one probe specific to CDKN2A gene and/or at least one nucleic acid primer pair specific to CDKN2A gene, and (ii) at least one probe specific to FGFR3 gene and/or at least one nucleic acid primer pair specific to FGFR3 gene, and optionally, a leaflet providing guidelines to use such a kit.

13. The kit according to aspect 12, wherein the kit comprises at least 5, 10, 15, 20, 25, 30, 40 or 50 probes or primers specific to CDKN2A gene.

14. The kit according to aspect 13, wherein the kit comprises at least one probe or primer specific to a CDKN2A sequence selected from the sequences of SEQ ID NOs: 1 to 15.

15. The kit according to any of aspects 12 to 14, wherein the kit comprises at least one probe or primer specific to FGFR3 gene suitable to detect at least one FGFR3 mutation selected from the group consisting of R248C, S249C, P250R, G372C, S373C, Y375C, G382R, A391E, K652E, K652Q, K652M and K652T, and any combination thereof.

16. The kit according to any of aspects 12 to 15, further comprising at least one probe specific to chromosome 11p region and/or at least one nucleic acid primer pair specific to chromosome 11p region.

17. The kit according to any of aspects 12 to 16, further comprising at least one reference probe specific to a chromosomal region that is rarely altered in bladder cancer and/or at least one nucleic acid primer pair specific to a chromosomal region that is rarely altered in bladder cancer.

### REFERENCES

Aveyard et al. J Mol Diagn 2004; 6: 356-65.

Bakkar et al., Clin Chem. 2005 August; 51(8):1555-7.

Berggren et al. Clin Cancer Res 2003; 9: 235-42.

Billerey et al. Am J Pathol 2001; 158: 1955-9.

Cappellen et al., Nat Genet. 1999 September; 23(1):18-20.

Chapman et al. Clin Cancer Res 2005; 11: 5740-7.

Coombs et al. Anal Biochem 1990; 188: 338-43.

Florl et al. Lab Invest 2000; 80: 1513-22.

Hu et al. Breast Cancer Res. 2010, Mar. 8; 12(2):R18.

Labarcaet al. Anal Biochem 1980; 102: 344-52.

Michiels et al. Carcinogenesis. 2009; 30:763-8.

Mostofi et al. (1973) Histological Typing of Urinary Bladder Tumours. Geneva: World Health Organization.

Orlow et al. J Natl Cancer Inst 1995; 87: 1524-9.

Orlow et al. Am J of Pathol 1999; 155: 105-13.

Sobin et al. Cancer 1997; 80: 1803-04.

Sylvester et al., Eur Urol. 2006 March; 49(3):466-5.

van Oerset al. Clin Cancer Res 2005; 11: 7743-8.

van Rhijn et al. Eur J Hum Genet 2002; 10: 819-24.

Williamson et al. Human molecular genetics 1995; 4: 1569-77.

Zieger et al. Int J Cancer 2009; 125: 2095-103.

I claim:

1. A method for predicting clinical outcome of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative of a non-muscle invasive bladder cancer with FGFR3 activating mutation with a poor prognosis.

2. The method according to claim 1, wherein a poor prognosis is a shorter progression free survival and/or an increased metastasis occurrence and/or a decreased patient survival.

3. The method according to claim 1, wherein the loss-of-function mutation in CDKN2A gene is a hemizygous or homozygous deletion of the CDKN2A gene.

4. The method according to claim 1, further comprising detecting a hemizygous or homozygous deletion at chromosome 11p region.

5. The method according to claim 1, wherein the activating mutation in FGFR3 gene is selected from the group consisting of R248C, S249C, P250R, G372C, S373C, Y375C, G382R, A391E, K652E, K652Q, K652M and K652T, and a combination thereof.

6. The method according to claim 1, further comprising assessing at least one other cancer or prognosis marker or expression of proliferation marker.

7. The method according to claim 6, wherein said other cancer or prognosis marker is tumor stage, grade, number of tumors, prior recurrence rate, mitotic index, tumor size, HJURP expression level or HP1α expression level.

8. The method according to claim 6, wherein said expression of proliferation marker is Ki67, MCM2, CAF-1 p60 or CAF-1 p150.

9. A method for selecting a subject affected with a non-muscle invasive bladder cancer with an activating mutation in FGFR3 gene for an anti-tumoral therapy, or for determining whether a subject affected with a non-muscle invasive bladder cancer with an activating mutation in FGFR3 gene is susceptible to benefit from an anti-tumoral therapy, wherein the method comprises detecting a loss-of-function mutation in CDKN2A gene in bladder cancer cells from the subject, the presence of a loss-of-function mutation in the CDKN2A gene being indicative that an anti-tumoral therapy is required for the subject affected with the non-muscle invasive bladder cancer with an activating mutation in FGFR3 gene.

10. The method according to claim 9, wherein the anti-tumoral therapy is an adjuvant or neoadjuvant therapy.

11. The method according to claim 9, wherein the anti-tumoral therapy is immunotherapy.

12. The method according to claim 11, wherein said immunotherapy is Bacille Calmette-Guerin (BCG) therapy.

13. The method according to claim 7, wherein the anti-tumoral therapy is partial or radical cystectomy.

14. A method for providing information for determining follow-up strategy of a subject affected with a non-muscle invasive bladder cancer with an activating mutation in FGFR3 gene, wherein the method comprises detecting a loss-of-function mutation in CDKN2A gene in bladder cancer cells from the subject, the presence of a loss-of-function mutation in the CDKN2A gene being indicative that an intensive follow-up is required for the subject affected with the non-muscle invasive bladder cancer with an activating mutation in FGFR3 gene.

15. A kit (a) for predicting or monitoring clinical outcome of a subject affected with a non-muscle invasive bladder cancer; and/or (b) for selecting a subject affected with a non-muscle invasive bladder cancer for an anti-tumoral therapy, or for determining whether a subject affected with a non-muscle invasive bladder cancer is susceptible to benefit from an anti-tumoral therapy; and/or (c) for providing information for determining follow-up strategy of a subject affected with a non-muscle invasive bladder cancer, wherein the kit comprises (i) at least one probe specific to CDKN2A gene and/or at least one nucleic acid primer pair specific to CDKN2A gene, and (ii) at least one probe specific to FGFR3 gene and/or at least one nucleic acid primer pair specific to FGFR3 gene, and optionally, a leaflet providing guidelines to use such a kit.

16. The kit according to claim 15, wherein the kit comprises at least 5, 10, 15, 20, 25, 30, 40 or 50 probes or primers specific to CDKN2A gene.

17. The kit according to claim 15, wherein the kit comprises at least one probe or primer specific to a CDKN2A sequence selected from the sequences of SEQ ID NOs: 1 to 15.

18. The kit according to claim 15, wherein the kit comprises at least one probe or primer specific to FGFR3 gene suitable to detect at least one FGFR3 mutation selected from the group consisting of R248C, S249C, P250R, G372C, S373C, Y375C, G382R, A391E, K652E, K652Q, K652M and K652T, and any combination thereof.

19. The kit according to claim 15, further comprising at least one probe specific to chromosome 11p region and/or at least one nucleic acid primer pair specific to chromosome 11p region.

20. The kit according to claim 14, further comprising at least one reference probe specific to a chromosomal region that is rarely altered in bladder cancer and/or at least one nucleic acid primer pair specific to a chromosomal region that is rarely altered in bladder cancer.


SEQUENCE LISTING (15 sequences)

SEQ ID NO: 1 - DNA, 77 bp, Homo sapiens
cgccgtgtctcaagatcgatgaaatgcggttaaaatgatgaatagaaactctagggggacctcatatcgatagactc

SEQ ID NO: 2 - DNA, 71 bp, Homo sapiens
cctgatgtgccacacatctttgacctcaggtttctaacgcctgttttctttctgccctctgcagacatccc

SEQ ID NO: 3 - DNA, 67 bp, Homo sapiens
ggctctacacaagcttcctttccgtcatgccggcccccaccctggctctgaccattctgttctctct

SEQ ID NO: 4 - DNA, 61 bp, Homo sapiens
ctggatcggcctccgaccgtaactattcggtgcgttgggcagcgcccccgcctccagcagc

SEQ ID NO: 5 - DNA, 54 bp, Homo sapiens
ggggcaccagaggcagtaaccatgcccgcatagatgccgcggaaggtccctcag

SEQ ID NO: 6 - DNA, 61 bp, Homo sapiens; identical to SEQ ID NO: 4

SEQ ID NO: 7 - DNA, 58 bp, Homo sapiens
gctcttccgccagcaccggaggaagaaagaggaggggctggctggtcaccagagggtg

SEQ ID NO: 8 - DNA, 60 bp, Homo sapiens
ggcgtccccttgcctggaaagataccgcggtccctccagaggatttgagggacagggtcg

SEQ ID NO: 9 - DNA, 61 bp, Homo sapiens
tgaacgcactcaaacacgcctttgctggcaggcgggggagcgcggctgggagcagggaggc

SEQ ID NO: 10 - DNA, 73 bp, Homo sapiens
gctcccaggtacagctgtgttaagccttcatagatgagttctagaataggatgttgggtgcaatagataatta

SEQ ID NO: 11 - DNA, 73 bp, Homo sapiens
gactctcctccctgggatccagtaaactgactctaaacttaaaatcttacctaaaatcctggacctcaattca

SEQ ID NO: 12 - DNA, 55 bp, Homo sapiens
gcaggttcttggtgaccctccggattcggcgcgcgtgcggcccgccgcgagtgag

SEQ ID NO: 13 - DNA, 62 bp, Homo sapiens
atgggctagacacaaaggactcggtgcttgtcccagccaggcgccctcggcgacgcgggcag

SEQ ID NO: 14 - DNA, 73 bp, Homo sapiens
ggaagaggaaagaggaagaagcgctcagatgctccgcggctgtcgtgaaggttaaaaccgaaaataaaaatgg

SEQ ID NO: 15 - DNA, 64 bp, Homo sapiens
gaggggacatggagggggagaccggagagagaacgtacgccgaggagtcaggcggcgggatcaa